(19) United States
(12) Patent Application Publication
(10) Pub. No.: US 2024/0376546 A1
(43) Pub. Date: Nov. 14, 2024

(54) TARGETS OVEREXPRESSED ON THE SURFACE OF CANCER CELLS

(71) Applicant: University of Cape Town, Cape Town (ZA)

(72) Inventors: Darren Martin, Cape Town (ZA); Krupa Naran, Cape Town (ZA); Sinkala Musalula, Cape Town (ZA); Stefan Barth, Cape Town (ZA)

(73) Assignee: University of Cape Town, Cape Town (ZA)

(21) Appl. No.: 18/553,024

(22) PCT Filed: Mar. 29, 2022
(86) PCT No.: PCT/IB2022/052869

§ 371 Date: Sep. 28, 2023

(30) Foreign Application Priority Data
Mar. 29, 2021 (GB) 2104445.8

(51) Int. Cl. C12Q 1/6886

(52) U.S. Cl. CPC C12Q 1/6886; C12Q 2600/106; C12Q 2600/158

(57) ABSTRACT

The present invention relates to methods and systems for identification of differentially expressed targets for anticancer agents and/or identification of potential CSR-related off-target effects of anticancer agents in a subject. In particular, the methods and systems allow for the identification of anticancer drugs for the treatment of cancer in a subject which should not develop off-target effects.

### CROSS REFERENCE TO RELATED APPLICATIONS

This application is the U.S. national phase of International Patent Application No. PCT/IB2022/052869 filed on Mar. 29, 2022, which claims priority to United Kingdom Patent Application No. 2104445.8 filed on Mar. 29, 2021, the contents of which are incorporated herein by reference in their entirety.

### BACKGROUND OF THE INVENTION

The present invention relates to methods and systems for identification of differentially expressed targets for anticancer agents and/or identification of off-target effects of anticancer agents in a subject.

Among the most notable differences between cancer cells and healthy untransformed cells is the overexpression in cancer cells of particular cell surface receptor (CSR) proteins amongst others. CSR proteins traverse the plasma membrane to provide sensory links between the extracellular environment and cytosolic signalling pathways. Besides being exposed on the external surfaces of cells, alterations in the signalling pathways within which many CSRs function are directly involved in oncogenesis. CSRs are, therefore, often effective targets for immunodiagnosis or anticancer drugs and antibody-based anticancer therapies. Alterations in the expression of CSRs are very common during oncogenesis and can involve gene mutations, gene copy number changes and/or transcriptional changes.

CSRs that are currently targeted to treat tumours were initially identified as useful targets based on either their overexpression in tumours compared to adjacent healthy tissues or their mutational profiles in cancer cells. However, this approach to identifying credible targets has generally given little consideration to the magnitude of the differential expression levels of targeted CSRs on tumour cells in comparison to adjacent healthy tissue as well as the tissues of body organs that are not directly associated with the primary tumours under consideration.

Many clinical trials that aim to evaluate the efficacy of CSR targeted treatments ultimately fail due to the dose-limiting toxicity and unexpected side-effects. Many of these undesirable side-effects are likely a consequence of interactions between small molecule drugs or therapeutic antibodies and CSRs in healthy tissues. If so, treatment success rates could be improved, and off-target toxicity could be reduced by targeting CSRs that are both overexpressed in cancer cells and under-expressed in all healthy tissues.

### SUMMARY OF THE INVENTION

The present invention relates to methods and systems for identification of differentially expressed targets for anticancer agents and/or identification of potential CSR-related off-target effects of anticancer agents in a subject. In particular, the methods and systems allow for the identification of anticancer drugs for the treatment of cancer in a subject which should not develop off-target effects.

In a first aspect of the invention there is provided for a method of identifying an anticancer agent, the method comprising the steps of: firstly establishing which cell surface receptors (CSR's) occur on the surface of a cell from a cancerous organ or tissue obtained from a subject, secondly establishing which CSR's occur on the surface of a cell from a healthy part of the same organ or tissue in the subject, and then determining which of the CSR's are differentially expressed in the cancerous organ or tissue. Once the differentially expressed CSR's have been identified listing these CSR's as potential targets for anticancer therapy. Consequently, confirming that the differentially expressed CSR's are expressed at a higher level on the cancer cells as compared to the cells from the healthy part of the same organ or tissue in the subject or confirming that the differentially expressed CSR's are expressed at a higher level on the cancer cells as compared to their expression in other healthy organs or tissues in the subject. Subsequently, identifying one or more anticancer agents which target the one or more of the differentially expressed CSR's from the cancerous organ or tissue, provided that the anticancer agents do not target cells from a healthy part of the same organ or tissue and/or which anticancer agents do not target cells from another healthy organ or tissue. It will be appreciated that the anticancer agent preferentially binds to the differentially expressed CSR on the surface of a cell from a cancerous organ or tissue. The anticancer agent is deemed to be a systemic target (or ideal target) and is preferably administered to a subject systemically when it does not bind to a CSR on the surface of a cell from a healthy part of the same organ or tissue in the subject and/or when the anticancer agent does not bint to a CSR on the surface of a cell from a healthy organ or tissue. The anticancer agent is deemed to be a local target (or other target) and is preferentially administered locally to a tissue or organ of the subject when it does bind to a CSR on the surface of a healthy part of the same organ or tissue and/or a healthy organ or tissue.

In a first embodiment of the invention there is provide for providing an organ or tissue sample collected from a subject for use in establishing which cell surface receptors (CSR's) occur on the surface of a cell from a cancerous organ or tissue obtained from a subject.

In a second embodiment of the invention the presence or absence of a particular CSR on the surface of a cell is established through measuring the mRNA transcript levels encoding the CSR in the cell. It will be appreciated that the mRNA transcript level may be measured by any technique for measuring mRNA levels that are known in the art. Preferably, the mRNA levels will be measured by a technique selected from the group consisting of microarray, SAGE, blotting, RT-PCR, sequencing or quantitative PCR.

It will also be appreciated that the anticancer agent may be selected from any anticancer agent known in the art. In other words any agent which has known activity in the control of cancer. Preferably, the anticancer agent will be selected from the group consisting of a polynucleotide, protein, peptide or small molecule.

It will be further appreciated that the differentially expressed CSR in the cancerous organ or tissue is most preferably overexpressed relative to the expression of the CSR in a healthy part of the same organ or tissue in the subject and/or overexpressed relative to the expression of the CSR in a different healthy organ or tissue.

In a further embodiment of the invention the differentially expressed CSR in the cancerous organ or tissue is overexpressed by a log-fold difference as compared to a healthy organ, healthy tissue or healthy part of the cancerous organ or tissue.

In a preferred embodiment when the differentially expressed CSR is expressed at a log-fold difference of between 1 and 2 as compared to a cell from a healthy organ or tissue or as compared to a cell from a healthy part of the cancerous organ or tissue, then the anticancer agent would be suitable for local administration to the cancerous organ or tissue. This is due to the fact that the anticancer agent may have some off target effects.

Further, it will be appreciated that when the differentially expressed CSR is expressed on a cancerous cell at a log-fold difference of greater than 2 as compared to its expression on a cell from a healthy organ, healthy tissue or healthy part of a cancerous organ or tissue then the anticancer agent is suitable for systemic administration to a subject. This is due to the fact that the anticancer agent is highly unlikely to have or will not have off target effects. An anticancer agent which targets a differentially expressed CSR which is present in a cancerous organ or tissue will most preferably not be expressed in a healthy organ or tissue of the subject and/or be expressed in a healthy organ or tissue of the subject at a level below a log-fold difference of 2.

In a further preferred embodiment of the invention the subject is a human.

In yet another embodiment of the invention the presence of the CSR on the surface of a cell from a cancerous organ or tissue and the absence of the CSR on the surface of a cell from all healthy organs or tissues makes it a suitable target for the anticancer agent. It will be appreciated that the anticancer agent is selected based on its specificity to the differentially expressed CSR. This is in order to avoid off target effects in the subject.

In a second aspect of the invention there is provided for a method of predicting an off-target effect or the likelihood an off target effect of an anticancer agent. The method comprising training a machine learning model or algorithm using data, wherein the data includes (i) data from reported adverse events in subjects treated with the anticancer agent, wherein the adverse events are mapped to a specific organ or tissue, (ii) data of CSR's expressed on a cell surface of a healthy organ or tissue that was the subject of the adverse event, and/or (iii) data confirming whether the anticancer agent targets one or more of the CSR's expressed on the cell surface of the healthy organ or tissue, and predicting the likelihood of an off-target effect occurring in a healthy organ or tissue of a subject treated with the anticancer agent, by utilising the trained machine learning model or algorithm.

A “module”, in the context of the specification, includes an identifiable portion of code, computational or executable instructions, or a computational object to achieve a particular function, operation, processing, or procedure. A module may be implemented in software, hardware or a combination of software and hardware. Furthermore, modules need not necessarily be consolidated into one device.

In a first embodiment of the invention the presence of a CSR on the surface of a cell is established through measuring mRNA transcript levels in the cell.

In a second embodiment the anticancer agent is selected from the group consisting of a polynucleotide, protein, peptide or small molecule.

In a third embodiment of the invention the machine learning model or algorithm comprises a quadratic support vector machines regression and/or a squared exponential Gaussian process regression.

In a fourth embodiment of the invention an increased likelihood of an off-target effect informs whether or not a specific anticancer agent should be used for treatment.

In a third aspect of the invention there is provided for a system for predicting an off-target effect of an anticancer agent, wherein the system includes a predication model. The prediction module of this aspect comprises or incorporates a machine learning model or algorithm which is trained by data, wherein the data includes: (i) data from reported adverse events in subjects treated with the anticancer agent, wherein the adverse events are mapped to a specific organ or tissue, (ii) data of CSR's expressed on a cell surface of a healthy organ or tissue that was the subject of the adverse event, and/or (iii) data confirming whether the anticancer agent targets one or more of the CSR's expressed on the cell surface of the healthy organ or tissue. The prediction module is configured to predict the likelihood of an off-target effect occurring in a healthy organ or tissue of a subject treated with the anticancer agent, by utilising the trained machine learning model or algorithm.

In one embodiment, the system includes a communication module which is configured to receive or retrieve information on a specific anticancer agent via a communication network or communication link from a user, which is then used by the prediction module in order to predict the likelihood of an off-target effect occurring in a healthy organ or tissue of a subject, if the subject is treated with the specific anticancer agent, wherein the communication module is further configured to send information on the predicted likelihood back to the user via the communication network or communication link.

### BRIEF DESCRIPTION OF THE FIGURES

Non-limiting embodiments of the invention will now be described by way of example only and with reference to the following figures:

FIG. 1: The number of CSR transcripts upregulated between breast tumours vs normal breast and breast tumours vs all other normal tissues. Sixty-two transcripts are commonly upregulated between the two sets of comparisons.

FIG. 2: Bar graphs showing the number of differentially expressed CSR transcripts between each pairwise comparison of the PAM50 breast cancer subtype (x-axis). The two bar graphs are plotted for the upregulated transcripts between each comparison (left) and downregulated transcripts between each comparison (right).

FIG. 3: A plot of the confusion matrix. The diagonal cells correspond to observations that are correctly classified. The off-diagonal cells correspond to incorrectly classified observations. Both the number of observations and the percentage of the total number of observations are shown in each cell. The column to the far right shows the precision (or positive predictive value rate) (top value in the cell) and the false discover rate (bottom value). The row at the bottom of the plot shows the recall (or true-positive rate) (top value) and the false-negative rate (bottom value). The cell in the bottom right of the plot shows the overall accuracy.

FIG. 4: The ROC-AUC (Receiver Operated Characteristic-Area Under the Curve) for Normal-like breast cancer. The ROC curves shows the true-positive rate versus false-positive rate for predictions of the PAM50 classes of breast cancer that were made by the trained classifier. The dot on the plots shows the values of the false positive rate and the true-positive rate of the trained classifier toward predicting the PAM50 subtype of breast cancer using CSRs.

FIG. 5: The ROC-AUC for HER2-positive breast cancer. The ROC curves shows the true-positive rate versus false-positive rate for predictions of the PAM50 classes of breast cancer that were made by the trained classifier. The dot on the plots shows the values of the false positive rate and the true-positive rate of the trained classifier toward predicting the PAM50 subtype of breast cancer using CSRs.

FIG. 6: The ROC-AUC for Luminal-A breast cancer. The ROC curves shows the true-positive rate versus false-positive rate for predictions of the PAM50 classes of breast cancer that were made by the trained classifier. The dot on the plots shows the values of the false positive rate and the true-positive rate of the trained classifier toward predicting the PAM50 subtype of breast cancer using CSRs.

FIG. 7: The ROC-AUC for Luminal-B breast cancer. The ROC curves shows the true-positive rate versus false-positive rate for predictions of the PAM50 classes of breast cancer that were made by the trained classifier. The dot on the plots shows the values of the false positive rate and the true-positive rate of the trained classifier toward predicting the PAM50 subtype of breast cancer using CSRs.

FIG. 8: The ROC-AUC curve for Basal-like breast cancer. The ROC curves shows the true-positive rate versus false-positive rate for predictions of the PAM50 classes of breast cancer that were made by the trained classifier. The dot on the plots shows the values of the false positive rate and the true-positive rate of the trained classifier toward predicting the PAM50 subtype of breast cancer using CSRs.

FIG. 9: Comparison between the number of differentially expressed CSR transcripts between each pair of PAM50 subtypes of breast cancer. The two plots show the comparison made for the TCGA primary tumours, whereas the two bottom plots show the comparison made for the GDSC breast cancer cell lines. The plots on the left column show the upregulated transcripts between each comparison and that on the right shows the downregulated transcripts between each comparison.

FIG. 10: Comparison of the drug-response profiles to CSR-targeting anticancer drugs between the cell lines that expressed higher transcript levels of the drug targets and those with lower transcript levels of the target. Each bar indicates the t-value calculated using the Welch test. The bars are coloured based on the level of statistical significance. Light grey bars denote statistically significant (p-value<0.05) increased response to the drug for the cell lines overexpressing the targets compare to those under expressing the drug target. Grey bars denote no statistically significant difference in the drug response. Black bars denote statistically significant lowered response to the drug. The comparisons were made across each drug represented in the GDSC database.

FIG. 11: Comparison of the drug-response profiles to CSR-targeting anticancer drugs between the cell lines that expressed higher transcript levels of the drug targets and those with lower transcript levels of the target. Each bar indicates the t-value calculated using the Welch test. The bars are coloured based on the level of statistical significance. Light grey bars denote statistically significant (p-value<0.05) increased response to the drug for the cell lines overexpressing the targets compare to those under expressing the drug target. Grey bars denote no statistically significant difference in the drug response. Black bars denote statistically significant lowered response to the drug. The comparisons were made across drugs that are grouped based on their target CSRs.

FIG. 12: Distribution of adverse events that are reported in the clinical trial of breast cancer for drugs that target particular CSRs. The group are segregated by the types of drug that are used to treat breast cancer patient: (1) Those that target highly expressed CSRs (the ideal targets) in breast tumours and (2) those that do not. The median of proportion individuals that experience adverse event is reported for each bar graph.

FIG. 13: Highlight table showing the number of breast cancer patients that experience a particular adverse event across all our classification of clinical trials which is based on the expression of CSR that are used as drugs cancers.

FIG. 14: Box plot displaying the typical values of the reported adverse event affecting a particular tissue for the drug dasatinib and the predicted response, and any possible outliers. The central mark indicates the median, and the bottom and top edges of the box are the 25th and 75th percentiles, respectively. The whiskers extend from the boxes to the most extreme data points that are not considered outliers, whereas outliers are shown individually using the “+” symbol.

FIG. 15: Flow diagram showing the overall study method.

FIG. 16: Schematic layout of a system in accordance with the invention.

FIG. 17: Example of ideal targets acute myeloid leukemia (AML). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 18: Example of other targets acute myeloid leukemia (AML). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 19: Example of ideal targets adrenocortical carcinoma (ACC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 20: Example of other targets adrenocortical carcinoma (ACC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 21: Example of ideal targets bladder urothelial carcinoma (BUC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 22: Example of other targets bladder urothelial carcinoma (BUC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 23: Example of ideal targets brain lower grade glioma (LGG). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 24: Example of other targets brain lower grade glioma (LGG). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue These are targets that can be targeted for treatment by local administration of an anticancer agent.

FIG. 25: Example of ideal targets breast invasive carcinoma (BIC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 26: Example of other targets breast invasive carcinoma (BIC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 27: Example of ideal targets cervical squamous cell carcinoma and endocervical adenocarcinoma (CSCC ECA). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 28: Example of other targets cervical squamous cell carcinoma and endocervical adenocarcinoma (CSCC ECA). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue These are targets that can be targeted for treatment by local administration of an anticancer agent.

FIG. 29: Example of ideal targets colon adenocarcinoma (CAA). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 30: Example of other targets colon adenocarcinoma (CAA). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 31: Example of ideal targets esophageal carcinoma (EC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 32: Example of other targets esophageal carcinoma (EC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 33: Example of ideal targets glioblastoma multiforme (GBM). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 34: Example of other targets glioblastoma multiforme (GBM). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 35: Example of ideal targets kidney chromophobe (KC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 36: Example of other targets kidney chromophobe (KC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 37: Example of ideal targets kidney renal clear cell carcinoma (RCCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 38: Example of other targets kidney renal clear cell carcinoma (RCCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 39: Example of ideal targets kidney renal papillary cell carcinoma (RPCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 40: Example of other targets kidney renal papillary cell carcinoma (RPCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 41: Example of ideal targets liver hepatocellular carcinoma (HCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 42: Example of other targets liver hepatocellular carcinoma (HCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 43: Example of ideal targets lung adenocarcinoma (LAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 44: Example of other targets lung adenocarcinoma (LAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 45: Example of ideal targets lung squamous cell carcinoma (LSCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 46: Example of other targets lung squamous cell carcinoma (LSCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 47: Example of ideal targets lymphoid neoplasm diffuse B cell lymphoma (DLBCL). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 48: Example of other targets lymphoid neoplasm diffuse B cell lymphoma (DLBCL). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 49: Example of ideal targets ovarian serous cyst adenocarcinoma (OSCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 50: Example of other targets ovarian serous cyst adenocarcinoma (OSCC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 51: Example of ideal targets pancreatic adenocarcinoma (PAAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 52: Example of other targets pancreatic adenocarcinoma (PAAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 53: Example of ideal targets prostate adenocarcinoma (PrAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 54: Example of other targets prostate adenocarcinoma (PrAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 55: Example of ideal targets all sarcomas (SAR). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 56: Example of other targets all sarcomas (SAR). Example of Other targets of Sarcomas. The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 57: Example of ideal targets stomach adenocarcinoma (SAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 58: Example of other targets stomach adenocarcinoma (SAC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 59: Example of ideal targets testicular germ cell tumour (TGCT). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 60: Example of other targets testicular germ cell tumour (TGCT). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 61: Example of ideal targets thyroid carcinoma (TC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 62: Example of other targets thyroid carcinoma (TC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 63: Example of ideal targets uterine carcinosarcoma (UCS). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 64: Example of other targets uterine carcinosarcoma (UCS). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 65: Example of ideal targets uterine corpus endometrial carcinoma (UCEC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 66: Example of other targets uterine corpus endometrial carcinoma (UCEC). The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

FIG. 67: Example of ideal targets of Skin Cutaneous Melanomas. The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by systemic administration of an anticancer agent.

FIG. 68: Example of other targets of Skin Cutaneous Melanomas. The bar graph at the top shows the difference in the mRNA expression levels of various genes between the tumours cells verse the same healthy tissue. The bar graph at the bottom shows the difference in the mRNA expression levels of various genes between the tumours cells verses all other healthy tissue. These are targets that can be targeted for treatment by local administration of an anticancer agent.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

Breast cancer is characterised by varied responses to different anticancer therapies, and these therapies produce a myriad of off-target effects. Front line treatment for early-stage breast cancer involves surgical tumour excision, typically accompanied by one or more modes of adjuvant therapy for the promotion of pathological complete response (pCR) (Recht et al., (1985), Fisher et al., (1998), Park et al., (2000)). Adjuvant therapies which have, to date, been validated for tumour-reducing potential include radiation therapy (localized), chemotherapy (systemic) and an increasing range of targeted therapies (Gerber (2008)). Adjuvant therapies administered leading up to surgery (neoadjuvant therapy) have been shown to reduce surgical margins and contribute to pCR post-surgery in many types of breast cancer (Liu et al., (2010)). Where metastasis is suspected, localized treatment is followed by systemic treatment to pursue remaining malignant cells. Regardless of pCR, the most commonly used systemic modality, being chemotherapy, is notoriously indiscriminate in its destruction.

It was hypothesised that for drugs that target cell surface receptors (CSRs), the different responses of tumours and the adverse events produced by these drugs may be attributed to variations in the transcriptional landscapes of CSRs in both breast tumours and healthy tissues. Using data from a variety of sources, the inventors compared the CSR transcriptional landscapes of different breast tumours and a range of different non-diseased human tissues. The inventors demonstrated an association between the responses to drug perturbation of breast cancer cell lines and the transcription levels of their targeted CSRs. Important differences in the CSR transcriptional landscapes of PAM50 primary breast tumour subtypes and the CSR transcriptional landscapes of breast cancer cell lines, were identified which will likely impact the accuracy of drug response predictions. Applying clinical trial data, the inventors exposed a link between the expression levels of CSR genes in healthy tissues and adverse reactions of patients to anticancer drugs. Altogether, this approach allows for the isolation of the most suitable CSR target(s) among the expressed transcripts, solely based on the measured dose-responses of cell lines to anticancer agents, the CSR transcriptional landscape in health patient tissues, and reported adverse responses of patients to drugs targeting CSRs. This might have significant implications for the future of precision medicine and immunotherapy by providing corresponding ideal target-based companion diagnostics and immunotherapeutics.

The inventors identified such CSRs in the context of finding targets for breast cancer diagnosis and treatment. Specifically, the present invention provides a platform for hypothesis generation and a framework for selecting which CSRs should be targeted to minimise the probability of undesirable treatment side-effects. Data was mined and integrated from multiple public resources and subjected to statistical methods, machine learning and predictive modelling to investigate the probable off-target toxic effects of targeting a variety of different CRSs; including many that are currently targeted and for which actual toxicity effects have been measured. Several explicit assumptions regarding the relationships between both drug action in the context of drug responses and off-target toxicity, and the transcriptional landscapes of the breast cancer cells and those of healthy body tissues, were made and these concepts were evaluated using data from thousands of high-quality laboratory measurements.

The present application relates to a bioinformatics approach, to investigate the relationship between the responses of cancer cell lines to a drug that targets specific CSRs to the transcription levels of the CSRs in those cell lines. Furthermore, the present invention evaluates the association between the adverse (or off-target) effects of CSR targeted drugs that are used to treat breast tumours and the transcriptional landscapes of the targeted CSRs in various healthy body tissues. Overall, the computational approach adopted revealed the link between drug action and the expression of CSRs in breast tumours, an insight that has been confirmed across many cancers of other tissues including acute myeloid leukemia, adrenocortical carcinoma, urothelial carcinoma, lower grade glioma, cervical squamous cell carcinoma, colon adenocarcinoma, oesophageal carcinoma, glioblastoma multiforme, renal clear cell carcinoma, renal papillary carcinoma, hepatocellular carcinoma, lung adenocarcinoma, lung squamous cell carcinoma, diffuse large cell lymphoma, ovarian serous cystadenocarcinoma, pancreatic adenocarcinoma, prostate adenocarcinoma, sarcoma, cutaneous melanoma, stomach adenocarcinoma, testicular germ cell tumours, thyroid carcinoma, uterine carcinoma, as well as uterine corpus endometrial carcinoma, and which provides a framework for improving the criteria by which drug targets are selected.

“Cells” are the basic structural and functional units of a living organism. In higher organisms, such as animals, cells with similar structure and function usually assemble into “tissues” or “organs” that perform specific functions. Thus, tissue includes similar cell aggregates and surrounding intercellular material such as epithelial tissue, connective tissue, muscles, nerves. “Organs” may be composed of different types of tissue, and fully differentiated structural and functional units in higher organisms specialised for some specific functions, such as the kidney, heart, brain, liver etc. Thus, by “specific organ, tissue or cell” is meant herein to include any specific organ and to include cells and tissues found in that organ.

The term “cell from a healthy organ or tissue”, as used herein, refers to a cell which is not affected by aberrant expression and/or abnormal proliferation, and does not derive from an organ or tissue or part of an organ or tissue that is cancerous.

A “cancer” is any unwanted growth of cells that does not provide a physiological function. In general, a cancer cell is a cell that deviates from its normal cell division control, in other words, its growth is not regulated by normal biochemical and physiological influences in the cell environment. Thus, “cancer” is a general term for diseases characterised by abnormal uncontrolled cell proliferation. In most cases, cancer cells proliferate to form clonal cells that are malignant. Cell masses or tumours can usually invade and destroy surrounding normal tissues. Cancer cells can spread through their lymphatic system or bloodstream from their original site to other parts of the body in a process known as “metastasis”. Many cancers are found to be resistant to treatment and are often fatal. Examples of cancer include, without limitation, transformed and/or immortalised cells in various organs and tissues.

Various aspects of the invention involve identifying agents (i.e. “anticancer agents”) which can be used to diagnose and treat cancers. In this context, treatment may be carried out so as to provide a variety of outcomes. For example, treatment may: (i) provoke an immune reaction that is effective to inhibit or ameliorate the growth or proliferation of a cancer, (ii) inhibit the growth or proliferation of cancer cells or tumours, (iii) cause remission of a cancer, (iv) improve quality of life, (v) reduce the risk of recurrence of a cancer, (vi) inhibit metastasis of a cancer, or (vii) improve patient survival rates in a patient population especially when combined with the corresponding companion diagnosis to identify the patients best responding to a CSR targeting therapy. In this context, extending the life expectancy of a patient, or patient population, means to increase the number of patients who survive for a given period of time following a particular diagnosis. In some embodiments, treatment may be of patients who have not responded to other treatments, such as patients for whom a chemotherapy or surgery has not been an effective treatment. A patient having a genetic or lifestyle predisposition to cancer of a certain tissue or organ may be treated with an anticancer agent.

A “cell from a cancerous organ or tissue” refers to cells from a part of an organ or tissue which contain cancer cells. It is understood that a cancer cell is a cell that exhibits abnormal proliferation and divides relentlessly, thereby forming a solid tumour or a non-solid tumour. These cells may be collected directly or surgically removed from an animal or human subject. Although the source organ or tissue is not limited, examples include: adipose tissue, adrenal glands, bladder, blood, blood vessels, bone marrow, brain, breast, cervix uteri, colon, oesophagus, fallopian tube, heart, kidney, liver, lung, muscle, nerve, ovary, pancreas, pituitary gland, prostate gland, skin, small intestine, spleen, stomach, testis, thyroid, uterus and vagina.

As used here in a “target” refers to a cell surface receptor which occurs on the surface of a cell from a cancerous organ or tissue.

As used herein “other target” refers to CSRs whose mRNA expression levels show a logfold difference of at least 2 on tumour cells in comparison to the healthy normal cells the tumour cells are originating from but still would show background expression other healthy organs or tissues.

Whereas an “ideal target” refers to a CSR whose mRNA expression levels show a log fold difference of at least 2 on tumour cells in comparison to the healthy normal cells that the tumour cells originate from, as well as with a log fold difference of at least 2 on tumour cells in comparison to other healthy organs or tissues. In other words, there is at least more than double the expression of the mRNA of a particular CSR in a cancerous cells or tissue, as compared to a healthy cell or tissue from the same or different cell or tissue.

The term “off-target effect” refers to the effects that can occur when an anticancer agent, such as a drug, binds to a target (proteins or other molecules in the body) other than those for which the anticancer agent, such as a drug, was meant to bind. This can lead to unexpected side effects that may be harmful to the subject being treated. Off-target activity is biological activity of a drug that is different from and not at that of its intended biological target. It most commonly contributes to adverse effects.

In the present invention, the term “off-target effect” means that anticancer agents target cell surface receptors of cells from a healthy organ or tissue. Such off-target effects may have detrimental effects on the cells from the healthy organ or tissue. It is desirable that anticancer agents or anticancer drugs do not show the so-called off-target effect in clinical use. In order to avoid or limit off-target effects, the present invention relies on log fold differences in expression of the CSRs on tumour cells and tissue in comparison to healthy organs and tissues. This is in order to ensure that the specific cell surface receptors being targeted by an anticancer agent are preferentially bound to the diseased cells but not to healthy organs or tissues in the subject. If an agent is identified as an “other target” this would imply that it would be suitable for local treatment of a tumour, in order to reduce off-target effects. If identified as “ideal target” this would imply that the agent would be suitable for systemic treatment or administration, as it would be expected to show no off-target effects. It is fundamentally important that anticancer agents, including polypeptides, nucleic acids, carbohydrates, lipids, receptor ligands, antibodies, small molecule compounds and any combination thereof, used in the treatment of cancer do not have or have reduced off-target effects as this may lead to potentially deleterious side effects.

The term an “anticancer agent” refers to any agent which is effective in the treatment of malignant, or cancerous, disease. In a preferred embodiment of the present invention an anticancer agent is an agent which specifically targets a CSR. There are several major classes of anticancer agents; these include alkylating agents, antimetabolites, natural products, and hormones. Additionally, there are a number of agents that do not fall within the aforementioned classes, but which demonstrate anticancer activity and thus are used in the treatment of malignant disease. The term chemotherapy is frequently refers to the use of chemical compounds as anticancer agents to treat cancer.

An “anticancer agent” can include any general anticancer drugs currently used in cancer therapy, as well as new anticancer drugs to be developed in the future. As used herein, the term “anticancer agent” refers to any agent that binds to a target cell and which does not bind to a noncancerous cell. The binding of the anticancer agent to the target cell will ordinarily occur via a cell surface receptor. Exemplary anticancer agents include, for example, a polypeptide, a nucleic acid, a carbohydrate, a lipid, a receptor ligand, an antibody, a small molecule and any combination thereof.

The anticancer agents of the invention may be used to treat cancer in a subject. It will be appreciated that treating a disease, disorder, condition or cell population includes therapy and prophylactic treatment on an acute short-term basis and on a chronic long-term basis.

The term “pharmaceutically acceptable” refers to properties and/or substances which are acceptable for administration to a subject from a pharmacological or toxicological point of view. Further “pharmaceutically acceptable” refers to factors such as formulation, stability, patient acceptance and bioavailability which will be known to a manufacturing pharmaceutical chemist from a physical/chemical point of view.

The “suitable forms” of the anticancer agents may be combined with “pharmaceutically acceptable carriers” and other elements known in the art in order to ensure efficient delivery of the active pharmaceutical ingredient to a subject.

By “pharmaceutically acceptable carrier” is meant a solid or liquid filler, diluent or encapsulating substance which may be safely used for the administration of the extract, pharmaceutical composition and/or medicament to a subject.

The term “effective amount” in the context of preventing or treating a condition refers to the administration of an amount of the active pharmaceutical ingredient in a pharmaceutical compound to an individual in need of treatment, either a single dose or several doses of the pharmaceutical compound may be administered to a subject.

The exact dosage and frequency of administration of the effective amount will be dependent on several factors. These factors include the individual components used, the formulation of the anticancer agent, the condition being treated, the severity of the condition, the age, weight, health and general physical condition of the subject being treated, and other medication that the subject may be taking, and other factors as are known to those skilled in the art. It is expected that the effective amount will fall within a relatively broad range that can be determined through routine trials.

Toxicity and therapeutic efficacy of anticancer agents of the invention may be determined by standard pharmaceutical procedures in cell culture or using experimental animals, such as by determining the LD50 and the ED50. Data obtained from the cell cultures and/or animal studies may be used to formulate a dosage range for use in a subject. The dosage of any anticancer agent of the invention lies preferably within a range of circulating concentrations that include the ED50 but which has little or no toxicity and little or no off-target effects. The dosage may vary within this range depending upon the dosage form employed and the route of administration utilised. For anticancer agents of the present invention, the therapeutically effective dose may be estimated initially from cell culture assays.

The term “cell surface receptor” as used herein refers to any molecule displayed on the surface of a cell and available for binding by therapeutic compounds which contact the surface of the cell. “Cell surface receptors” may also be referred to as membrane receptors or transmembrane receptors and are receptors that are embedded in the plasma membrane of cells. They act in cell signalling by receiving (binding to) extracellular molecules. “Cell surface receptors” are specialised integral membrane proteins that allow communication between the cell and the extracellular space. The extracellular molecules that bind to cell surface receptors may be proteins, such as hormones, neurotransmitters, cytokines, growth factors, cell adhesion molecules, drugs or nutrients. These extracellular molecules react with the “cell surface receptor” to induce changes in the metabolism and activity of a cell. In the process of signal transduction, ligand binding affects a cascading chemical change through the cell membrane. Cell surface receptors act as receptors for anticancer agents which may be used as therapeutic compounds.

The term “differentially expressed” refers to a measurement of gene and/or protein expression between at least two cells, wherein there is a difference in the amount of gene and/or protein product between the at least two cells. A gene is considered to be “differentially expressed” if an observed difference or change in read counts or expression levels between two experimental conditions is statistically significant. “Differential expression” is the biochemical processes that determines which genes are actively transcribed and translated into mRNA and proteins in a cell and under what conditions.

The following examples are offered by way of illustration and not by way of limitation.

### Example 1

### Analysis of CSR's Across Breast Tumours and Healthy Tissues

Transcriptome profiles of healthy tissues were accessed from three resources:
(i) The Genotype-Tissue Expression consortium—54 body tissues (Ardlie, K G., et al. (2015));
(ii) The Human Protein Atlas project—33 tissues (Pontén, F., et al. (2008)); and
(iii) The Functional Annotations of the Human Genome Project—57 tissues (Kawaji, H., et al. (2017)).

By collating these data, 101 unique transcriptome profiles were obtained for all major organs and various tissue types in the human body. A list was then compiled comprising CSRs using information from the literature, UniProt Knowledgebase (Uniprot (2017)), Surfaceome database (Bausch-Fluck, D., et al. (2015)), and the Gene Ontology Consortium (Gene Ontology Consortium (2015)) using the Gene Ontology term plasma membrane. This list of CSRs was used to extract only the mRNA transcription data of genes that are CSRs to which an unsupervised hierarchical clustering was applied to reveal clustering of healthy tissues based on their CSR expression (FIG. 15).

mRNA Expression of CSR Across Breast Tumours and Healthy Tissues

To establish the landscape expression of CSR across healthy tissues and breast cancer tissues. mRNA expression data collected and processed from 9,685 individuals across 31 healthy tissues expression by the GTEx and those of 1,079 breast cancer samples by the TCGA from the ARCHS4 database (Lachmann, A., et al. (2018), Weinstein, J N., et al. (2013). These datasets, which are directly comparable because they were re-processed using the same computation pipeline by the reCount2 project (Collado-Torres, L., et al. (2017) were used. A t-Distributed Stochastic Neighbourhood Embedding was applied to reveal the clustering pattern of breast cancer tumours and healthy tissues.

### Identification of the Ideal Drug Target CSR

The inventors hypothesised that anticancer drugs that target CSR are also likely to exhibit off-target adverse effects that are related to the expression of CSR in other healthy tissues. Therefore, the best drug target CSR would be those that are upregulated in the disease state compared to any other healthy body tissues. To identify such CSR targets, differential gene expression was performed using the negative binomial test, by comparing the CSR transcript between the breast tumour from TCGA and each of the healthy tissues from the GTEx project. The CSR transcripts that were found to be upregulated (adjusted p-value<0.05 and log 2 fold change>1) across all the breast cancer tissues versus healthy tissues comparisons (i.e., the intersection) were identified as the “ideal” drug and antibody targets.

Prediction of Breast Cancer Subtypes Using their CSR Transcription Data

Since breast tumour are subtyped using the PAM50 classification (Luminal A, Luminal B, Normal-like, basal and HER-2 positive) scheme, the inventors set to find out if they reproduce the tumour subtype using only mRNA transcripts of the CSRs. Using the reported breast cancer PAM50 subtypes within the TCGA dataset and their transcript levels of CSR genes, the inventors applied embedded feature selection method based on the boosted decision tree-based machine learning algorithm to identify 50 CSR features that were the most important predictors of the breast cancer PAM50 subtypes. Then an ensemble prediction model was trained by aggregating 20 decision trees using Random Undersampling Boosting, that was used to predict the breast cancer subtypes of the TCGA breast tumours into the Luminal A, Luminal B, Normal-like, basal and HER-2 positive subtypes.

### Influence of CSR Transcription on the PAM50 Subtypes Response to Drugs

To evaluate how the mRNA transcriptions of CSR may be related to the overall response of PAM50 subtypes of breast tumour to an anticancer drug, the inventors utilised the corresponding PAM50 subtype of breast cancer cell lines. Here, the dose-responses from the GDSC (Yang, W., et al. (2012)) of breast cancer cell lines that were previously classified into the four breast cancer subtypes by Dai et al. [47] and Aniruddha [48] were used. For each group of cell lines that belong to two different breast cancer subtypes, the inventors compared their drug-response to a particular anticancer drug for all 32 anticancer drugs using the student t-test with unequal variance assumed.

### Comparison of the CSR Transcription in Primary Tumours Versus Cancer Cell Lines

To evaluate the mRNA transcription of CSRs of the primary breast cancer PAM50 subtypes represented in the TCGA database with their corresponding PAM50 cancer cell lines that are represented within the GDSC database, the list of the differentially expressed transcript between each pair of PAM50 subtypes was compared. More specifically, first, the differentially expressed CSR transcripts were identified between each pair of PAM50 subtypes of the GDSC cancer cell lines using the Welch test. Then compared the list of differentially expressed CSRs between each pair of breast cancer PAM50 subtype (e.g., basal vs HER2 positive) of the primary tumours and those the corresponding cancer cell lines, (basal vs HER positive). Here, the inventors expected a concordant list of up- and down-regulated for each matched comparison.

### Association of Cell Line CSR Transcription and Drug Response

The breast cancer cell lines were classed into two categories for each drug-response comparison regardless of their breast tumour PAM50 subtype classification: 1) those that overexpress the CSR target of the drug and 2) those which under expression the target CSR for the drug. To dichotomise the cancer cell lines into these two groups, first, for each drug, the inventors retrieved the transcription profile of target across cell lines from the GDSC and applied z-normalisation to the profile. Then using a cut-off value of 1 standard deviation, the inventors categorised the cell lines with z-score values above 1 as those with higher drug target expression and the cell lines with a z-score less than −1 as those with lower drug target expression. The cell lines with z-scores that fall within −1 to 1 were excluded from the comparison on a drug-to-drug basis. To compare the mean drug-responses of these two groups of cell lines (high drug target expression and lower drug target expression groups), the inventors applied a Welch test to cell line's group the area under the curve of the dose-response curve.

### Validation of the Ideal Targets Using Reported Adverse Events

Data of breast cancer clinical trials that applied a single anticancer drug or antibody was retrieved (Zarin, D. A., et al. (2016)). Further, the inventors obtained the actual targets of each of the drugs applied in the clinical trials from the Pharos database (Nguyen, D-T., et al. (2017)) and Drug Gene Interaction Database (Cotto, K. C., et al. (2018)) to return only clinical trials that utilised drugs that target CSRs. The clinical trial records include information of participants of clinical trials, such as the treatment and adverse events that were experienced by each participant. Therefore, the inventors extracted information on the anticancer treatments used and the adverse events reported for each anticancer drug.

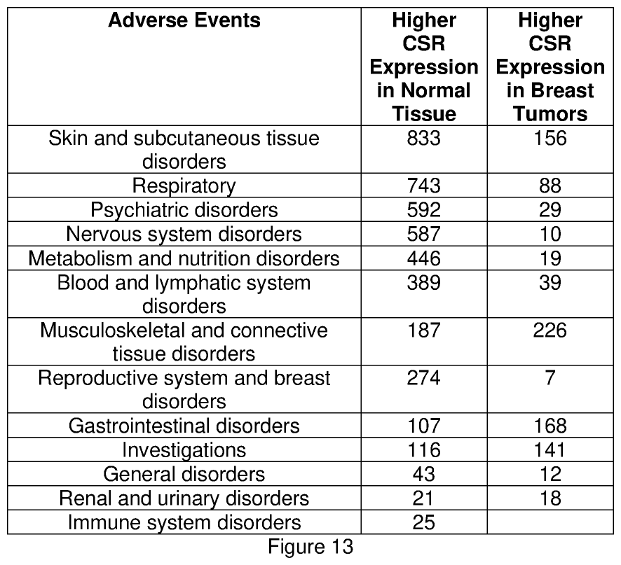

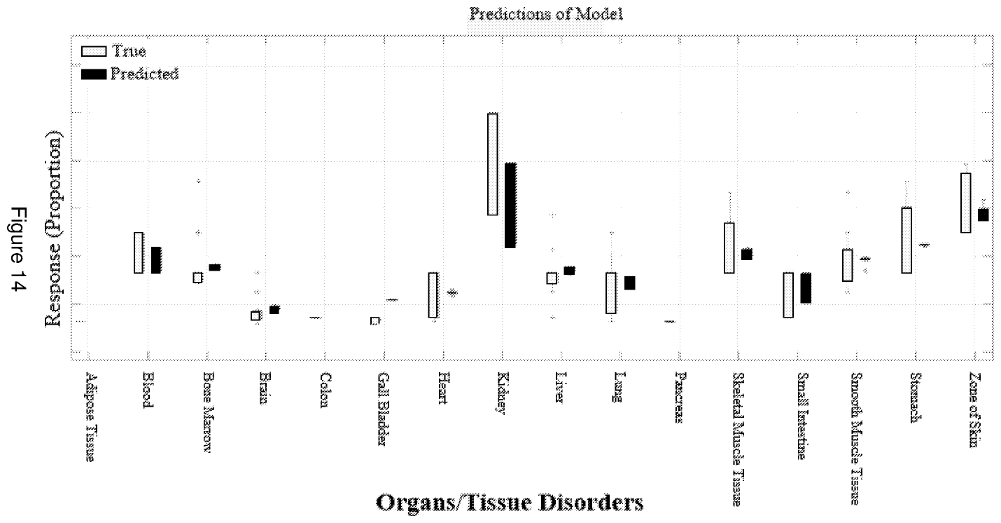

The inventors segregated the clinical trials into two sets: those that utilised CSRs that the inventors identified as the “ideal” targets (i.e., highly expressed in breast cancer compared to any other healthy body tissues) and those that utilised the “other targets”. The clinical trials that employed the “ideal targets” reported adverse events for 544 individual participants, whereas those that employed “other targets” reported adverse for 501 individual participants. Finally, the inventors compared the reported proportions and the actual number of individuals that experienced adverse events between these two categories of clinical trials. They found that for clinical trials that the “ideal targets” trials reported significantly fewer drug-associated adverse events than the “other targets” trials (Chi-square test, χ²=15.2, p-value 9.8×10-5; FIG. 12). Also, across various categories of adverse effects, a significantly higher proportion of patients in the “other targets” trials (median 4.8%) reported adverse effects than those in the idea targets trials (median=1.9%; rank-sum test statistic=186.5; p-value=0.0094) (FIG. 13).

### Prediction of the Adverse Events Using the CSR Transcription Levels of the Healthy Tissues

The inventors mapped adverse events reported in the clinical trials to particular body tissues: e.g., “Skin and subcutaneous tissue disorders” were ascribed to the skin, whereas “cardiac disorders” ascribed to the heart. Then the inventors annotated each of these adverse events to the healthy tissue expression of the CSR transcript levels that were obtained from the GTEx data, e.g., the row in the data that specifies the adverse events that occur in the heart due to some anticancer drug are related to the CSR expression of the healthy heart.

Further, for each of the drugs used in the clinical trial, the inventors had also obtained the drug target from the Pharos database and Drug Gene Interaction database to return only clinical trials that utilised drugs that target CSRs.

The present inventors used these data—adverse events ascribed to a particular tissue and the tissue CSR transcript levels of the drug target (use in the treatment and producing the adverse events)—to train a machine learning model to predict the adverse events in various healthy tissue. The inventors trained 20 different machine learning regression models including linear regression (using a simple linear model, interaction terms and the stepwise methods), decision trees regression (of various tree and leaf size), support vector machines regression (of various kernel scale, kernel function and box constraint), ensemble trees (boosted and bagged trees), and Gaussian process regression (of various kernel scale, kernel function and signal standard deviation and sigma). The inventors selected the two best performing regression models based on the 5-fold cross-validation accuracy: quadratic support vector machines model (root mean squared error=0.042) and the squared exponential Gaussian process regression (root mean squared error=0.043). The inventors then combined these two best performing models by training an ensemble machine learning algorithm based on quadratic support vector machine regression and squared exponential Gaussian process regression. This ensemble model was used to predict the adverse event which each particular anticancer agent would produce based on the CSR expression of the anticancer drugs targets in various healthy tissues.

### Statistical Analysis and Data Visualisation

All statistical analyses were performed in MATLAB 2019b. Fisher's exact test was used to assess associations between categorical variables. The independent sample Student t-test, Welch test and the one-way Analysis of Variance were used to compare continuous variables where appropriate. Statistical tests were considered significant at p<0.05 for single comparisons, whereas the p-values of multiple comparisons were adjusted using the Benjamini-Hochberg method. All the results and data were visualised either in MATLAB 20109b or Tableau version 2019.1.7.

### The Transcriptional Landscape of CSRs Across Breast Tumours and Healthy Tissues

To define patterns of CSR transcription across various normal tissues, the inventors retrieved and collated mRNA transcription data of 101 major body organs and tissues from the GTEx project, FANTOM project and the Human Protein Atlas databases. Upon filtering this data to retain only 1,140 CSR gene expression levels, hierarchical clustering was used to investigate variations in the expression of CSRs across different healthy organs and tissues. Similarly processed mRNA transcription data from breast cancer samples was obtained from the cancer genome atlas (TCGA) (1,091 samples) and compared with CSR mRNA transcription data from health tissues obtained from the GTEx project (9,658 samples including 218 samples from healthy breast tissue.

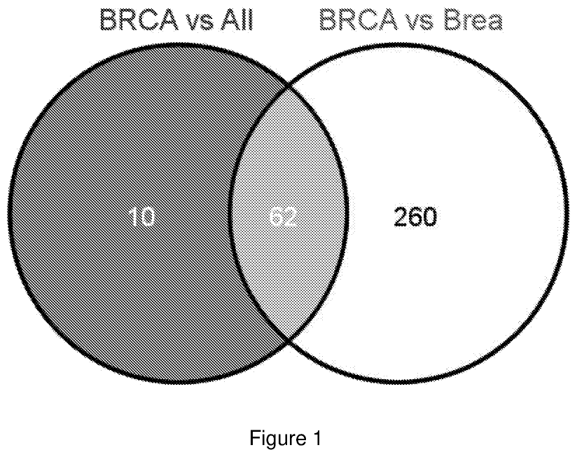

A total of 634 CSR transcripts were identified that were differentially expressed (log-2 fold-change>2 or <−2, and adjusted p-value<0.05) between the breast tumours and healthy breast tissue, and 581 CSR transcripts that were differentially expressed between breast tumours and healthy body tissues in general. Here, 322 CSR transcripts were identified that were more highly expressed in breast tumours than in healthy breast tissue (FIG. 1). Among the most significantly up-regulated transcripts were CEACAM6 (log 2FC=8.8), KCNJ (log 2FC=8.4) and CLDN6 (log 2FC=7.6). Furthermore, 72 CSR transcripts were identified that were significantly upregulated in breast tumours compared to non-breast healthy body tissues (FIG. 1). Among these were VTCN1 (log 2FC=7.0), LRRC (log 2FC=5.8) and SLITRK6 (log 2FC=5.5). The inventors found that only 62 transcripts were common between those that were: (1) upregulated in breast tumours relative to the healthy breast tissue and (2) upregulated in breast tumours relative to healthy non-breast tissues (FIG. 1). Included in these are transcripts for ERBB2, ERBB3, EPCAM, and IGFR.

Additionally, the inventors found that 511 CSR genes were significantly downregulated in breast tumours compared to healthy non-breast tissues. Interestingly, among these downregulated genes in breast tumours, 72 were significantly upregulated in breast tumours relative to healthy breast tissue. Among these transcripts that showed a discrepancy are well-known drug targets that are used in the treatment of breast cancer, including FGFR3, CD48 and CCR3. These findings indicated that even when drug targets are highly expressed in breast tumours relative to healthy breast tissues; the targeted CSR may be highly expressed in, potentially many, healthy tissues.

Natural inter-tissue variations in the expression of CSR proteins such as those highlighted above is expected to complicate the translation of CSR-binding anticancer molecules into useful therapeutics. Specifically, the presence of large numbers of targeted CSRs on the cells of healthy tissues is likely to be the primary cause of the dose-limiting toxic effects that are commonly associated with CSR targeted anticancer drugs.

Accordingly, the inventors hypothesised that such off-target toxic effects could be lessened by targeting CSRs that are expressed at higher amounts on cancer cells than they are on all healthy breast and non-breast tissue types. By comparing CSR transcript levels in breast tumours to those in all healthy tissue types, 26 mRNA transcripts were identified which, by being significantly more highly expressed in breast tumours than in any healthy tissue type, could potentially be targeted by anticancer drugs with minimal off-target cytotoxic effects.

### PAM50 Breast Cancer Subtypes Exhibit Distinct CSR Transcriptional Patterns

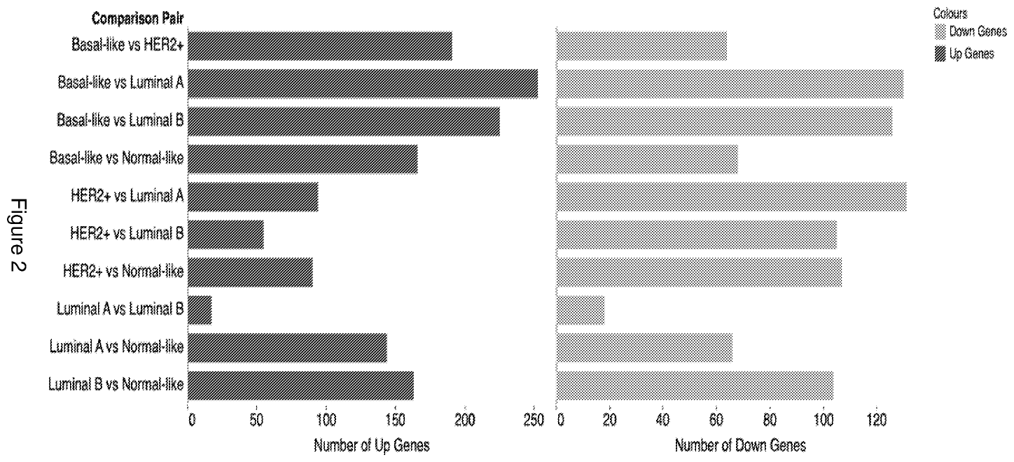

Breast tumours are classified into five molecular subtypes based on a 50-gene signature, called PAM50. These PAM50 subtypes are Luminal A, Luminal B, Normal-like, Basal-like and HER-2 positive. By comparing the CSR transcript levels between tumours annotated as belonging to the different PAM50 subtypes by the TCGA, the inventors found substantial differences in the transcript levels of various CSR genes between the subtypes (FIG. 2). The inventors identified that the highest number (323) of differentially expressed transcripts were between Basal-like and Luminal A breast tumours, and the fewest (32) between Luminal A and Luminal B breast tumours.

Since PAM50 subtyping of breast tumours is critical in the treatment and prognosis of breast cancer, the inventors sought to evaluate whether variations in CSR mRNA transcript levels alone could be utilised to classify breast cancer tumours accurately. PAM50 annotations that are provided within the TCGA database were used for each sample together with the sample's CSR transcription data to train a supervised machine learning model to classify tumours.

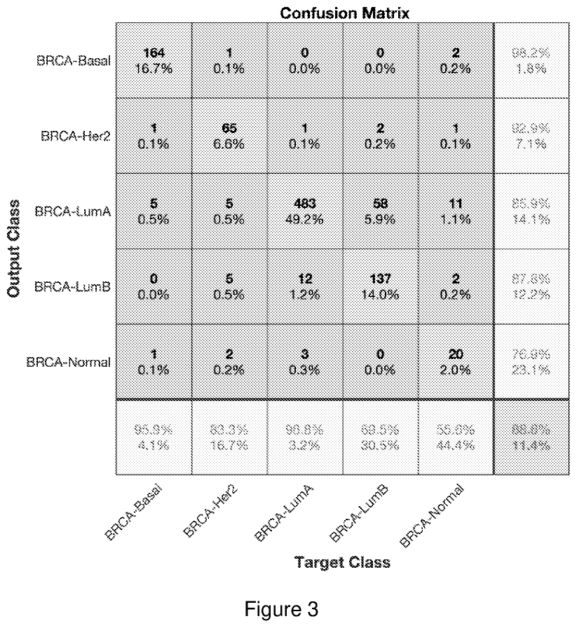

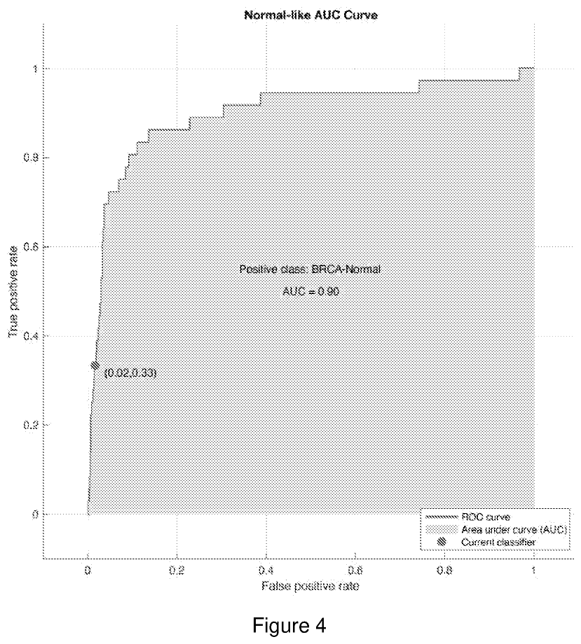

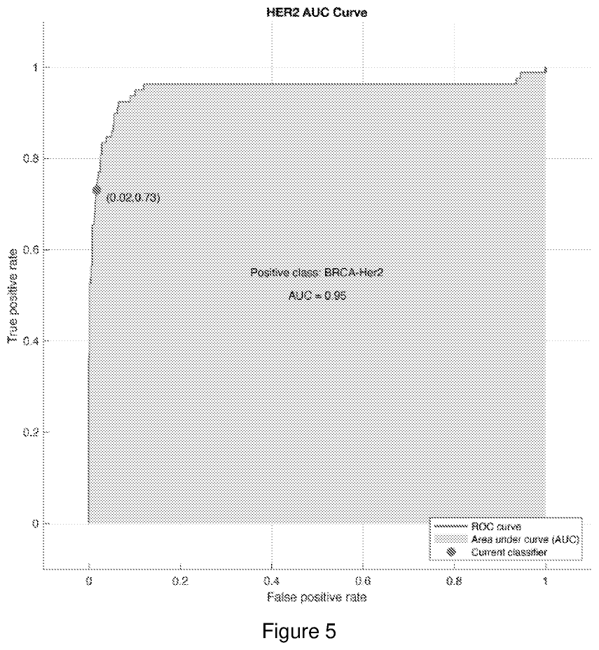

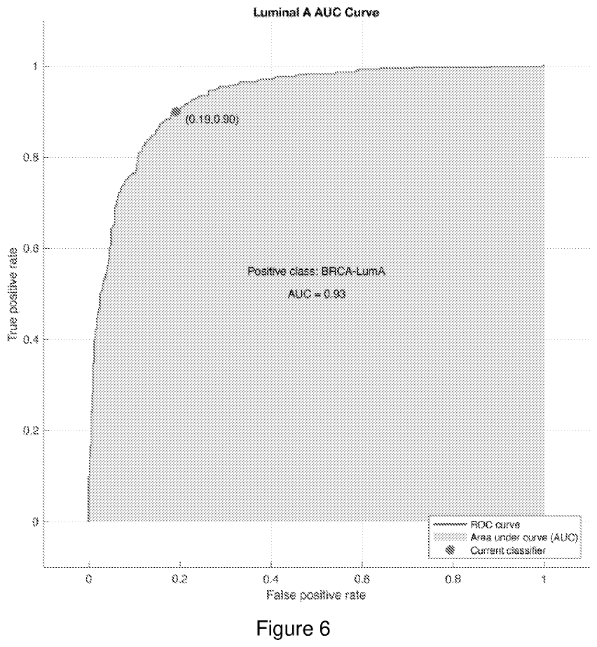

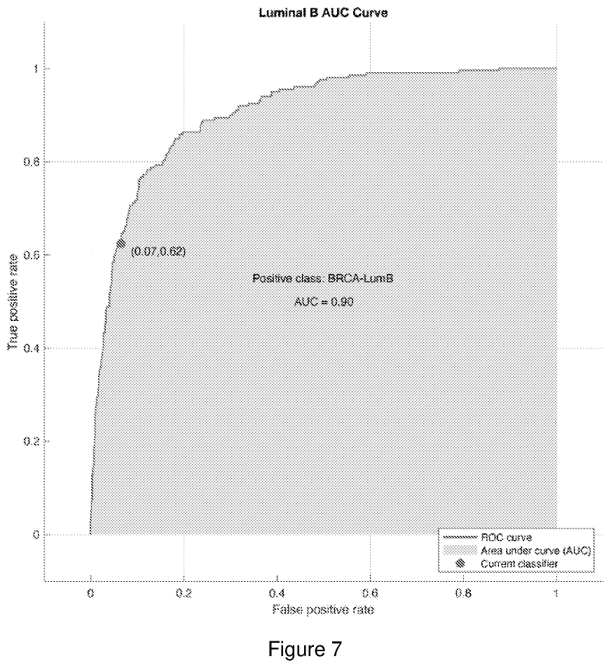

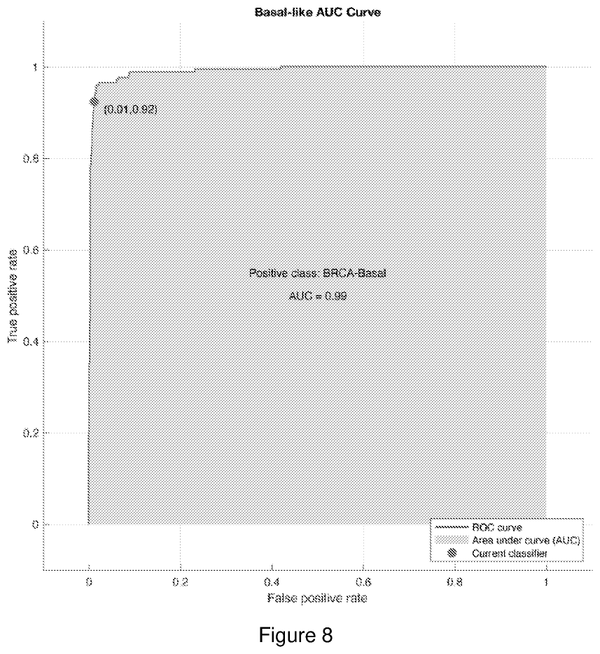

By applying an ensemble boosted decision tree model, the inventors found that they could accurately predict (average area under the curve of 93% and classification accuracy of 89%) the PAM50 subtype of breast tumour samples based exclusively on 1140 CSR transcript levels (FIG. 3-8). The model had positive predictive values of 85.9% for Luminal A, 87.8% for Luminal B, 76.9% for Normal-like, 98.2% for Basal-like and 92.9% for HER-2 positive breast cancer (FIG. 3-8). Overall, these results indicated that HER-2 and Basal-like breast tumours have the most distinctive CSR transcription profile, whereas Luminal A and Luminal B have transcription profiles that are the most difficult to differentiate between.

Drug Responses are Associated with the Transcriptional Levels of Targeted CSRs

The inventors sought to evaluate whether the response profiles of breast cancer cell lines to CSR targeted drugs differed in association with the PAM50 subtype classification of the cell lines. Using drug response data that is available from the GDSC for Luminal A, Luminal B, Basal-like and HER2 breast cancer cell lines, pairwise comparison of the mean drug-responses between cell lines of each breast cancer subtype to thirteen CSR targeting drugs were made. After correcting for multiple comparisons, it was found that, except for Basal-like versus Luminal A breast tumours (adjusted p-value=0.0079), the drug-response profiles of the different subtypes of cancer cell lines were not significantly different.

It was hypothesised that the transcriptional profiles of CSRs of the breast cancer cell lines that are represented within the GDSC database might differ from the transcriptional profiles of primary tumours (such as those represented within the TCGA database). If so, then it would be expected that, for a particular CSR targeting drug, drug-responses of the cell lines would vary between cell lines based primarily on differences between the expression levels of the targeted CSR.

To test this hypothesis, the inventors focused their analysis on CSR transcripts that are consistently differentially expressed between cell lines classified as belonging to different PAM50 subtypes.

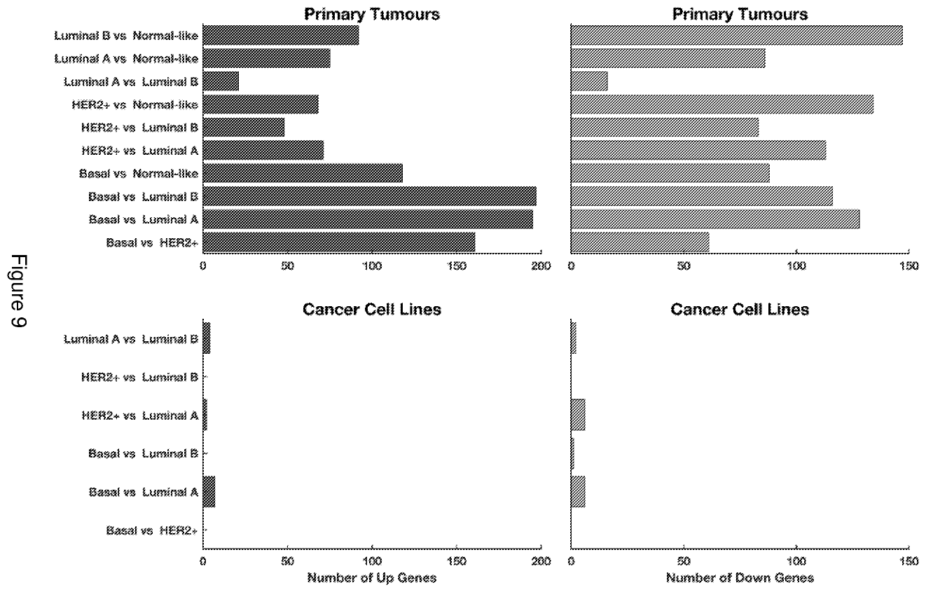

No CSR transcripts at significantly different levels (adjust p-values less than 0.05 and log 2 fold change higher than one or less than minus one) were found when comparing the Basal-like and HER2+ subtypes or the HER2+ and Luminal B subtypes. Only one CSR transcript with significantly different levels in the Basal-like and Luminal B subtypes of breast cancer cell lines was identified. Therefore, relative to primary tumours belonging to the different PAM50 subtypes—which display substantial differences in CSR expression (FIG. 9)—it appears as though breast cancer cell lines display far more uniform CSR expression patterns across the subtypes. This may explain, at least in part, the observed uniformity in responses to CSR targeted drugs across breast cancer cell lines belonging to different PAM50 subtypes.

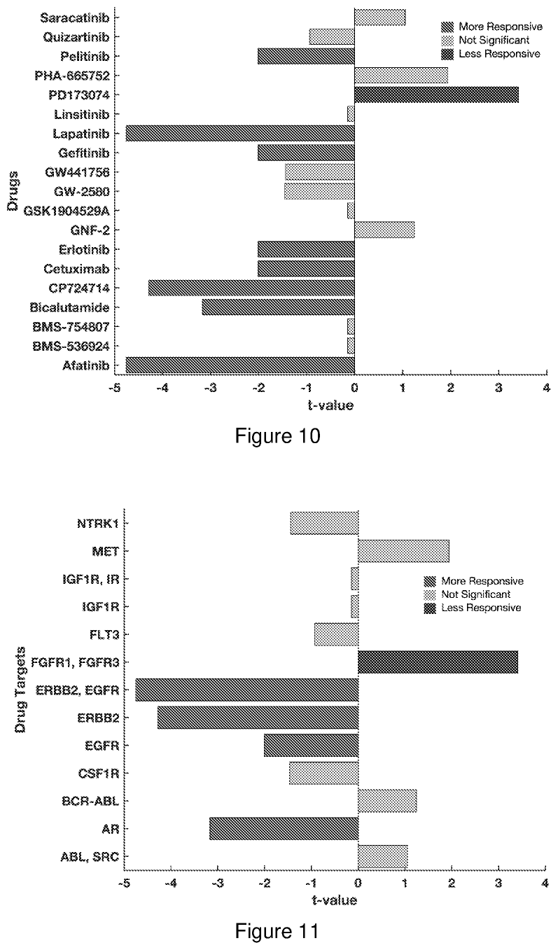

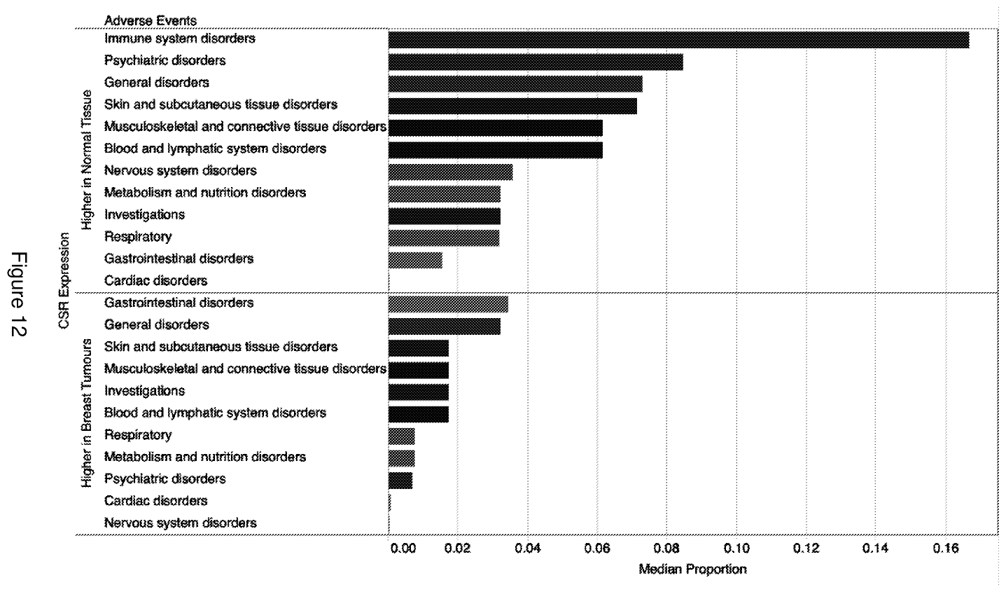

Therefore, rather than focusing on the PAM50 classifications of breast cancer cell lines when comparing responses to CSR targeted drugs, the inventors instead focused on the expression levels of particular drug-targeted CSRs in the cell lines and for each drug-response test simply divided the cell lines into higher and lower CSR expression categories. Remarkably, it was found that the drug-response profiles of 42% (8 of the 19 anticancer drugs) differed significantly between the higher and lower targeted CSR expression groups (FIGS. 10 & 11). Also, among these drug-response comparisons, 15/19 of the anticancer drugs displayed negative t-values (propensity towards higher efficacy in the higher group than in the lower group), which further confirmed that, for most of these anticancer drugs, drug efficacy is associated with the transcriptional levels of the CSRs that they target.

The mRNA Transcript Levels of CSRs in Healthy Tissues are Associated with Adverse Drug Events

The inventors hypothesised that the toxicity of CSR targeting drugs is associated with the CSR gene transcriptional levels in healthy tissues. Therefore, the inventors, examined breast cancer clinical drug trial data extracting information relating to the anticancer drug tested and reported adverse events which could be ascribed to drug toxicity in specific body tissues for which CSR transcription data was available.

Next, the clinical trials were segregated into two categories: (1) those involving drugs that target CSRs that are more highly expressed in breast tumours than in healthy body tissues, referred to as “ideal targets” (544 participants; FIGS. 12 & 13); and (2) those involving drugs that target any other CSRs, referred to as the “other targets” (501 participants). The proportions of individuals that experienced adverse events between these two clinical trial categories were then compared.

It was found that for clinical trials that the “ideal targets” trials reported significantly fewer drug-associated adverse events than the “other targets” trials (Chi-square test, χ²=15.2, p-value 9.8×10⁻⁵; FIG. 12). Also, across various categories of adverse effects, a significantly higher proportion of patients in the “other targets” trials (median 4.8%) reported adverse effects than those in the idea targets trials (median=1.9%; rank-sum test statistic=186.5; p-value=0.0094) (FIG. 13).

It is apparent, therefore that a positive association exists between adverse CSR-targeted drug responses and the expression levels of the targeted CSRs in healthy cells.

### Machine Learning Predicts the Adverse Drug Events Using the CSR Transcription Profiles of Drug Targets Across Body Tissues

Machine learning methods were used to determine whether the data from clinical trials could be used to predict the occurrence of adverse drug toxicity events in healthy tissues. Here, the inventors extracted information on tissue-level mRNA transcript measurements for CSRs that were targeted in published clinical drug trials which reported adverse events affecting healthy tissues. These data were then used to train a Gaussian process regression (Qinonero-Candela J. et al. (2007)) and support vector machine (Platt J C. (1999)) ensemble machine learning model and this trained model was used to predict adverse drug reactions using with an independent test set.

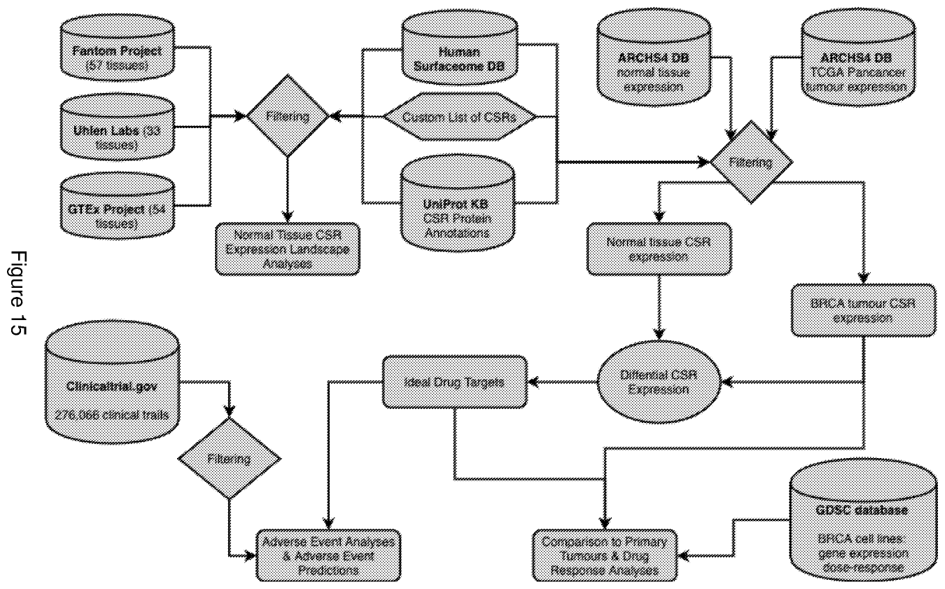

For each anticancer drug using tissue-level transcript abundances for the targeted CSR as inputs, the model accurately predicted, which healthy tissues would experience adverse events (R²=0.75). Furthermore, for each anticancer drug, the model accurately predicted the proportion of individuals that were likely to experience adverse events associated with a particular tissue (FIG. 14). For example, the inventors were able to show that using the ensemble machine learning model trained on the transcript levels of CSRs that are targeted by the drug, dasatinib (ABL, SRC, EPH, PDGFR, and KIT), the model predicted the proportion of patients exhibiting an adverse drug reaction (FIG. 14).

### Example 2

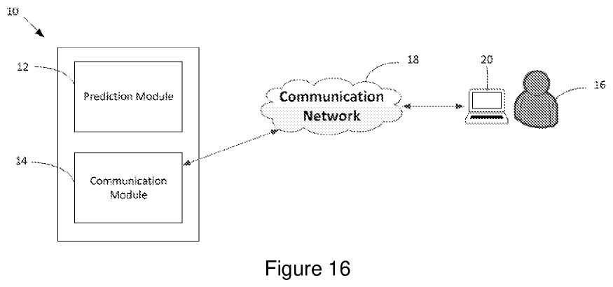

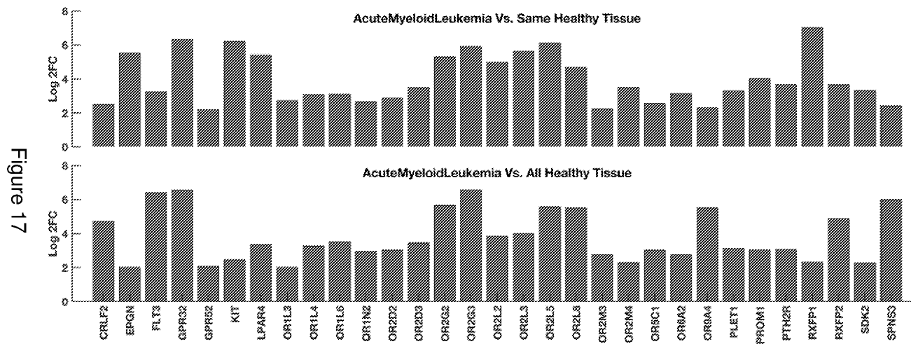

In one practical application, the machine learning model can be implemented within a system 10, in accordance with the invention, for predicting an off-target effect of an anticancer agent (FIG. 16). More specifically, the system 10 includes a prediction module 12 which comprises/incorporates the above-mentioned machine learning model (or algorithm). The system 10 also includes a communication module 14 which is configured to communicate with one or more users 16 (e.g. medical practitioners) via a communication network/link 18 (e.g. the Internet).

In practice, a user 16 would utilise a computer 20 or smart device (e.g. a smart phone or tablet) to send information on a specific anticancer agent via the communication network 18 to the communication module 14. In other words, the user 16 may be located remote from the communication module 14. The prediction module 12 then, in turn, utilises the machine learning model in order to predict the likelihood of an off-target effect occurring in a healthy organ or tissue of a subject treated with the specific anticancer agent. The communication module 14 then communicates the predicted likelihood back to the user 16 via the communication network 18.

In a slight variation, the prediction module 12 and communication module 14 can be implemented on a computer, smart device or another computing device (e.g. the computer 20), which is then used by a user 16. In this example, the communication module 14 would communicate with the user 16 via a user interface (e.g. displayed on a display screen of the computer or smart device), instead of sending information via a communication network.

### Example 3

### Analysis of CSR's Across Various Cancers Compared to Healthy Tissues

### Identification of the Ideal Targets

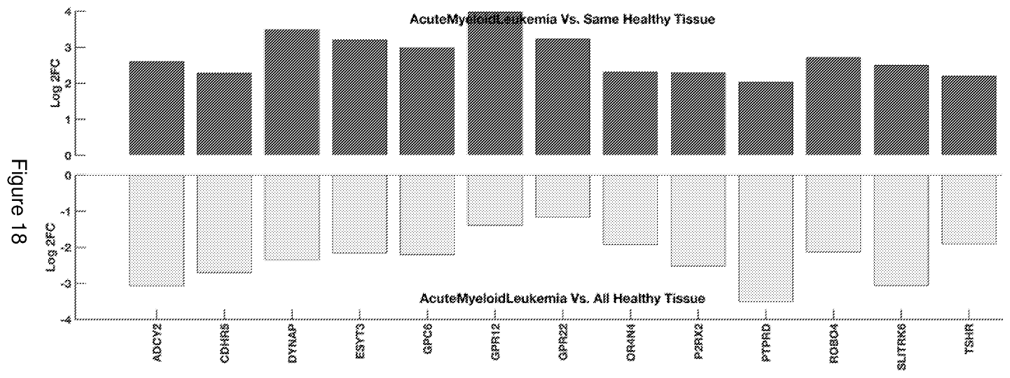

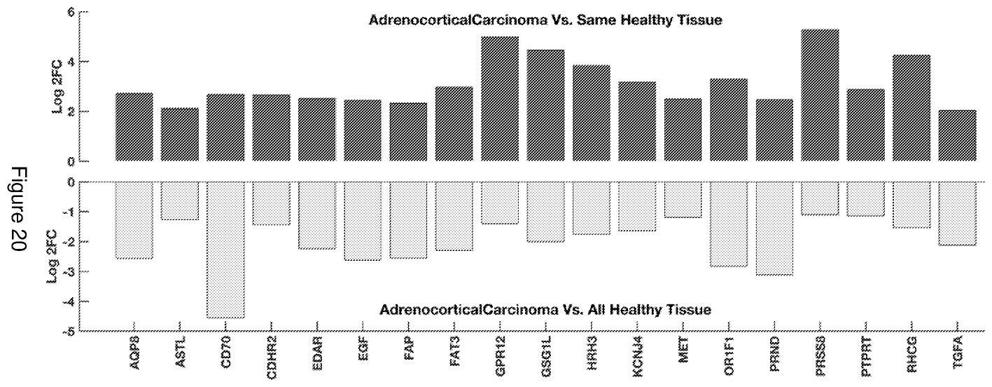

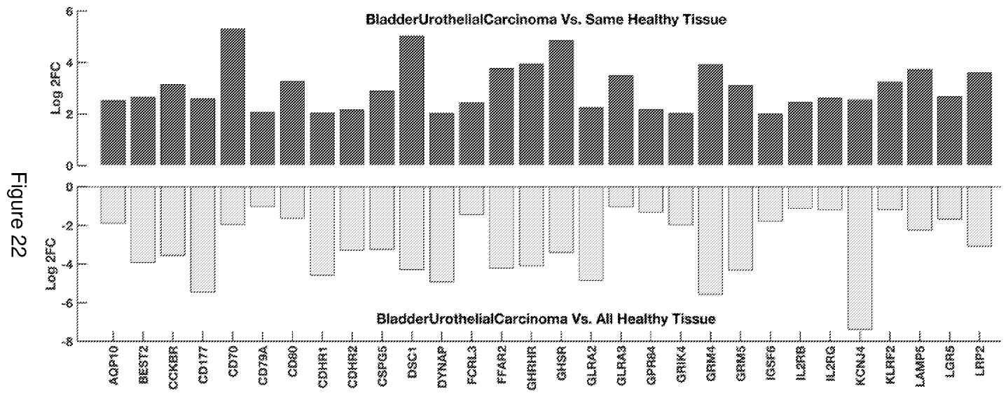

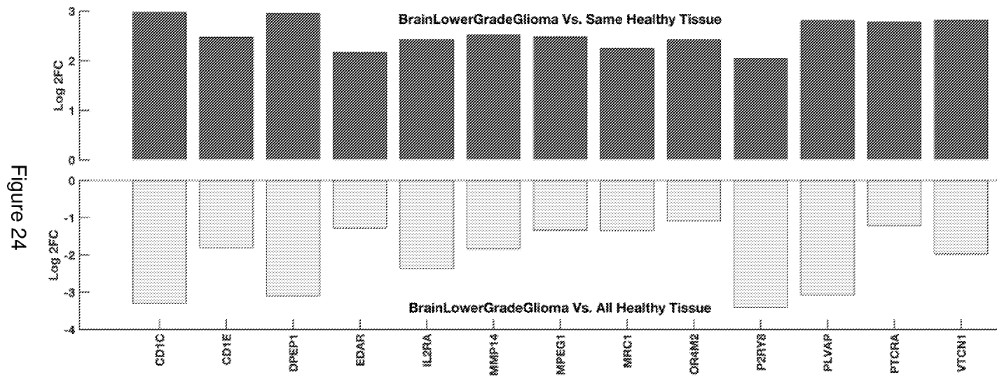

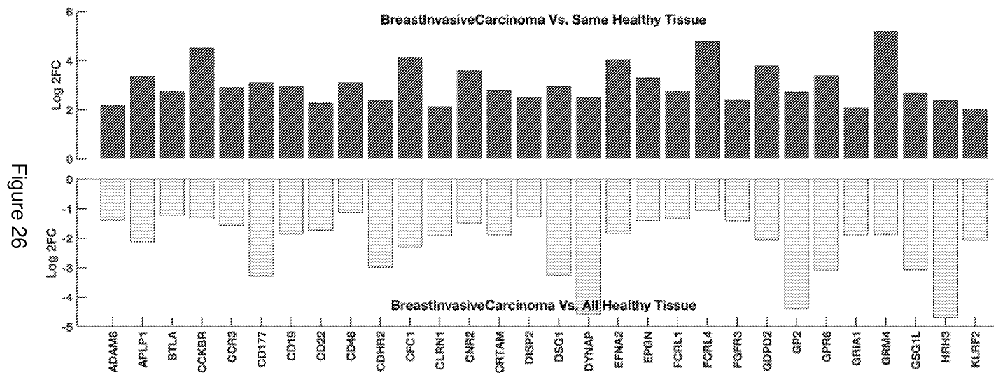

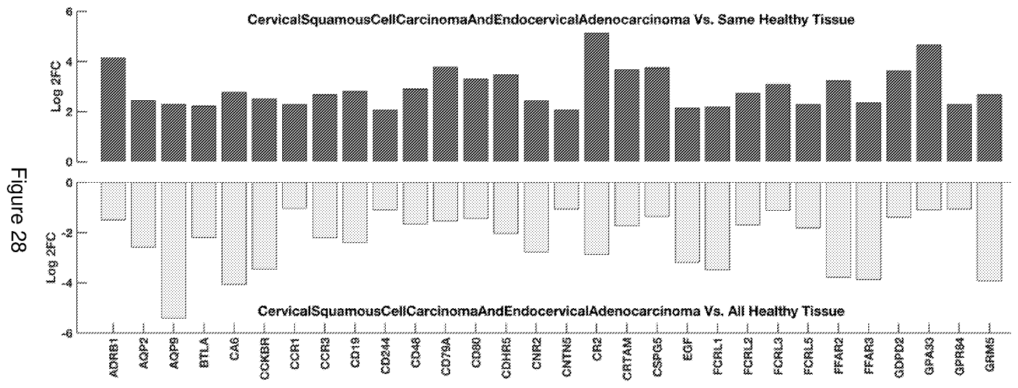

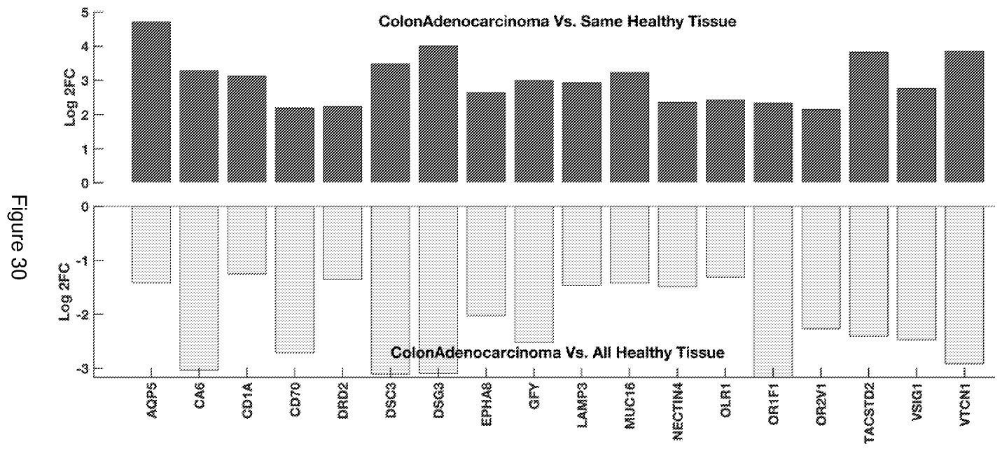

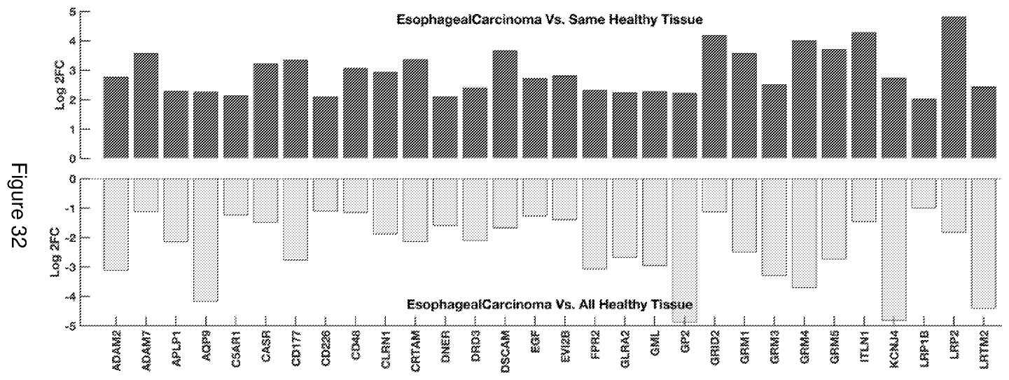

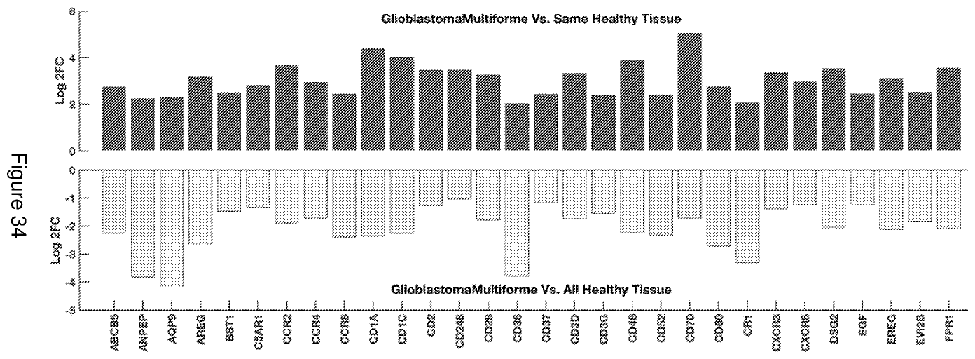

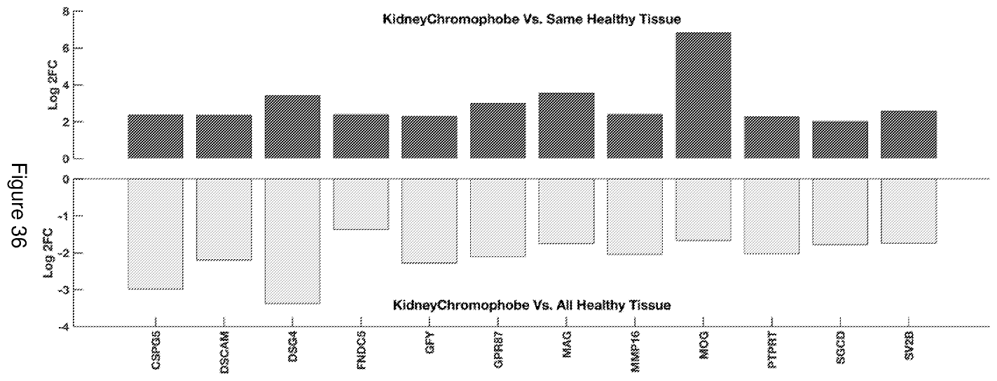

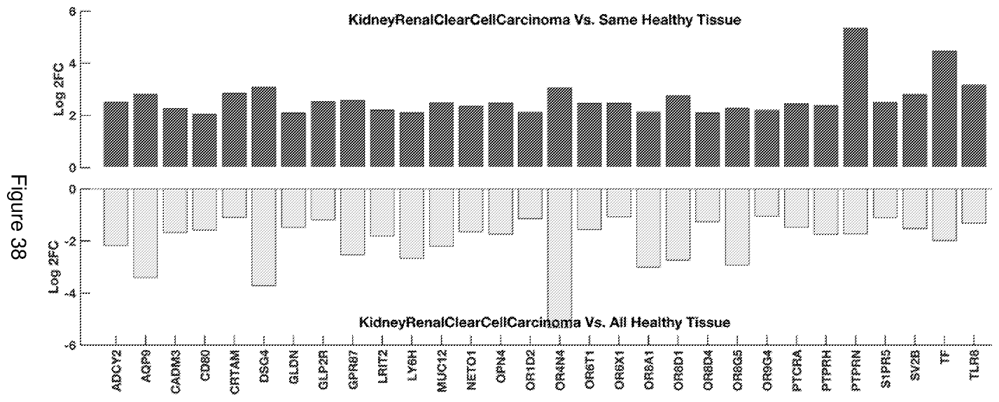

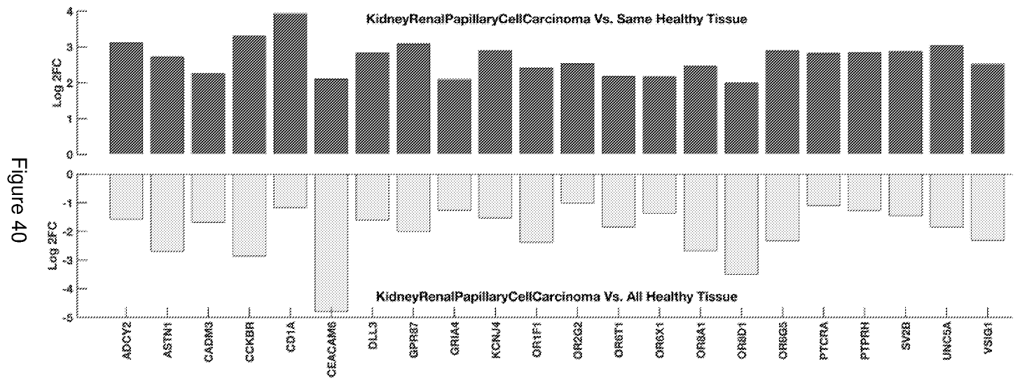

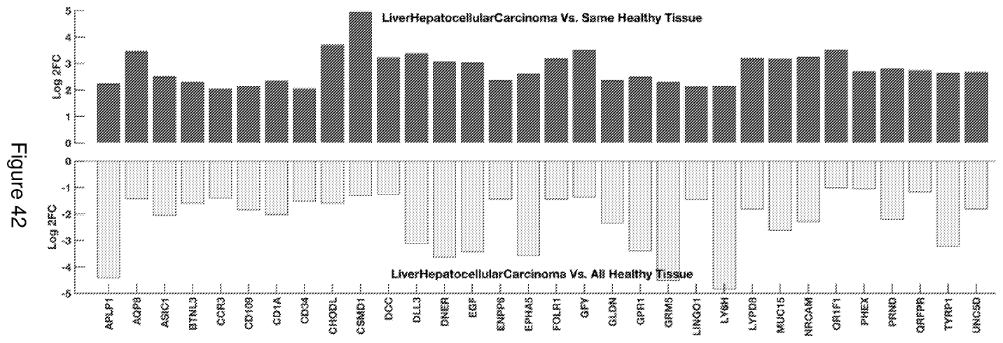

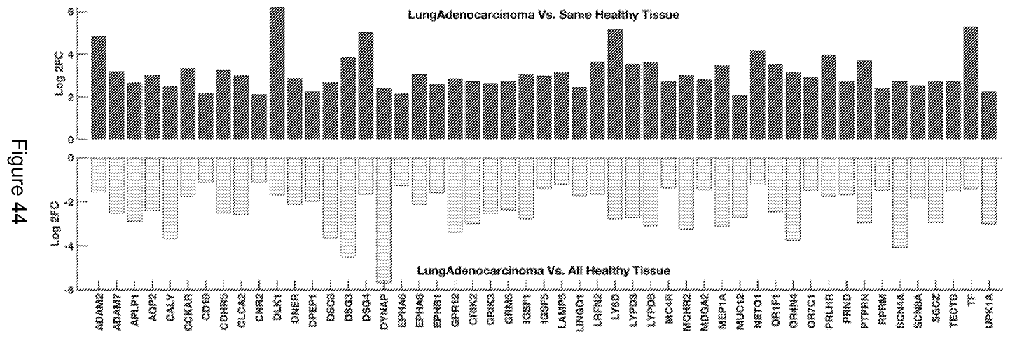

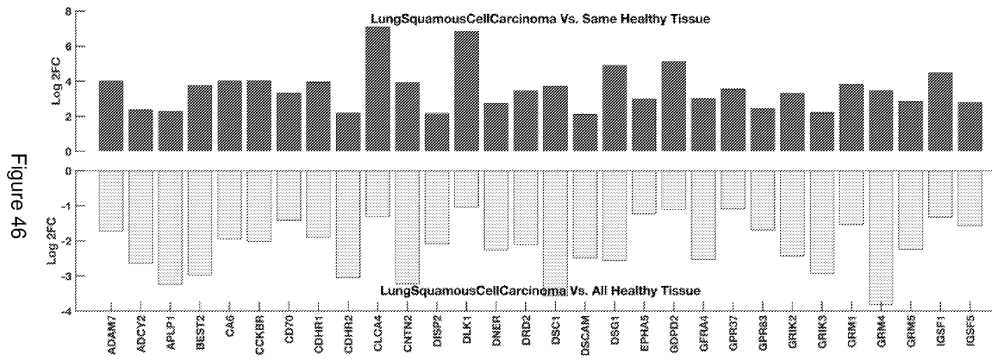

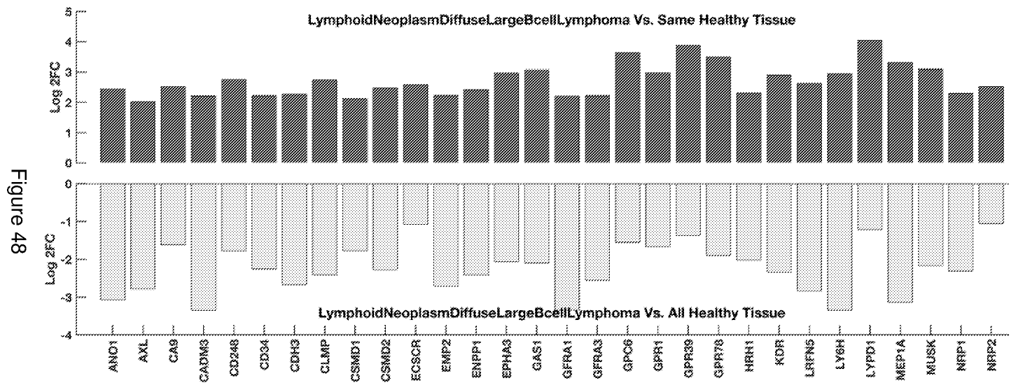

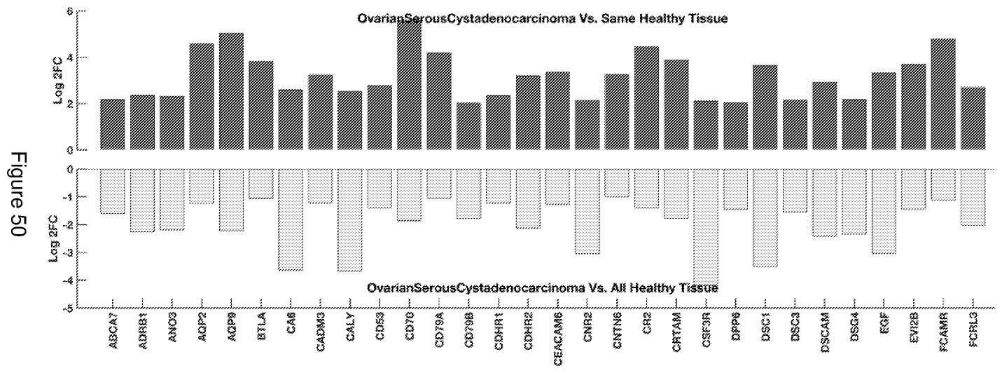

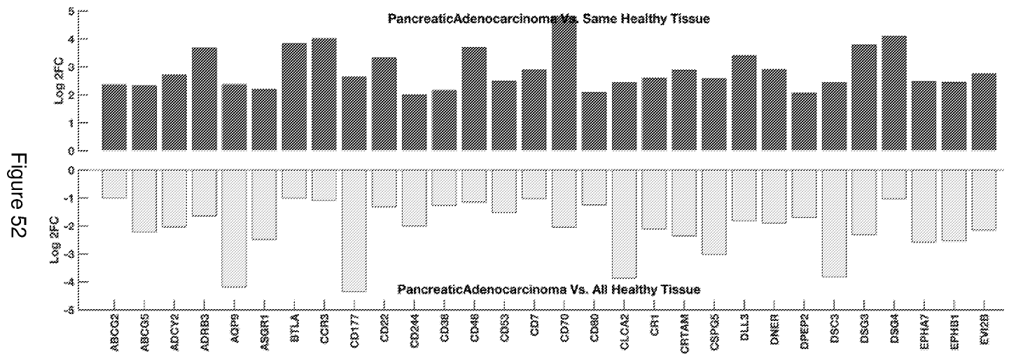

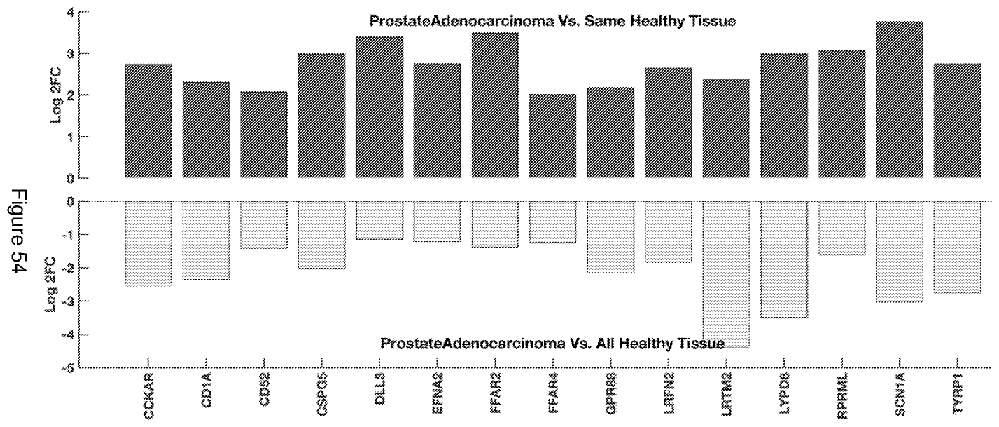

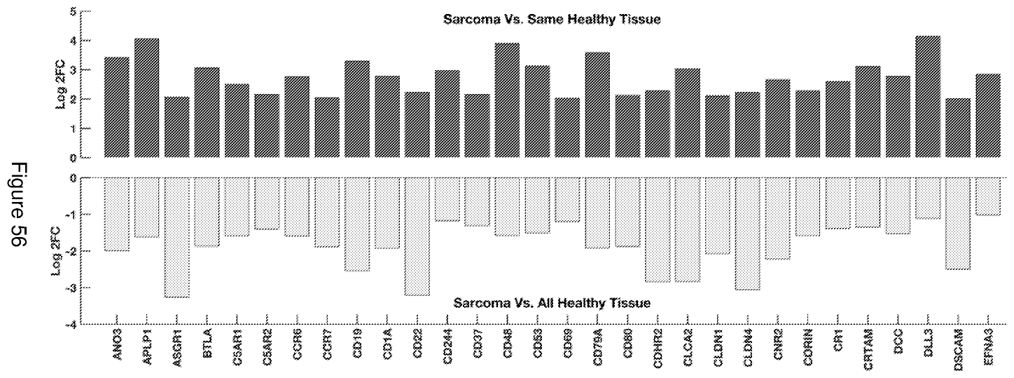

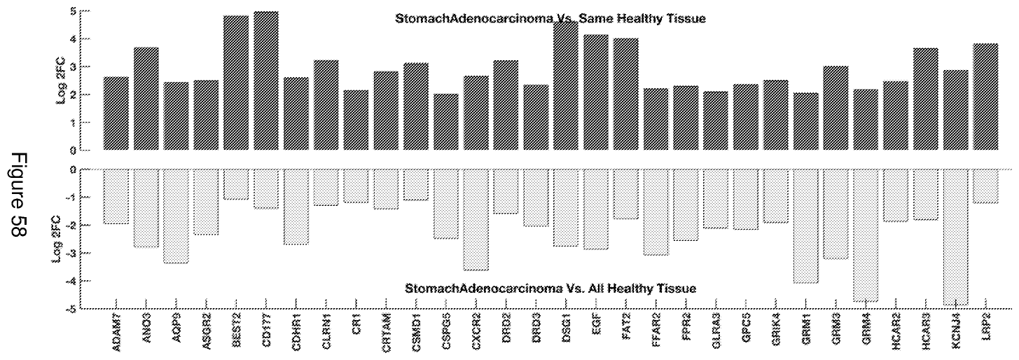

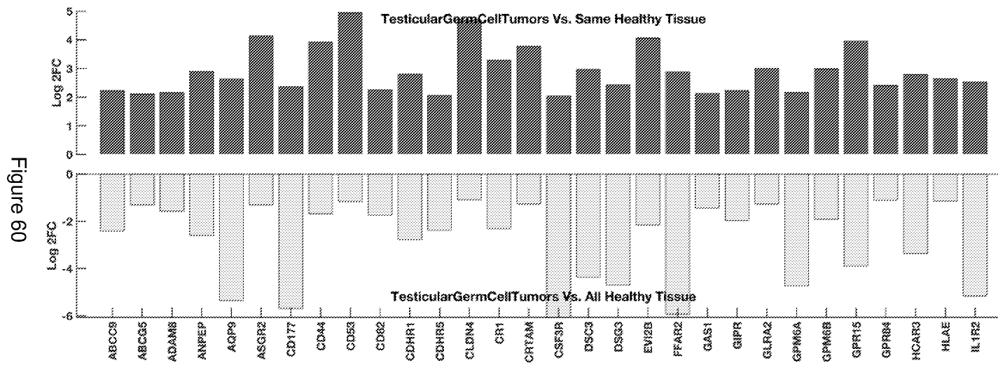

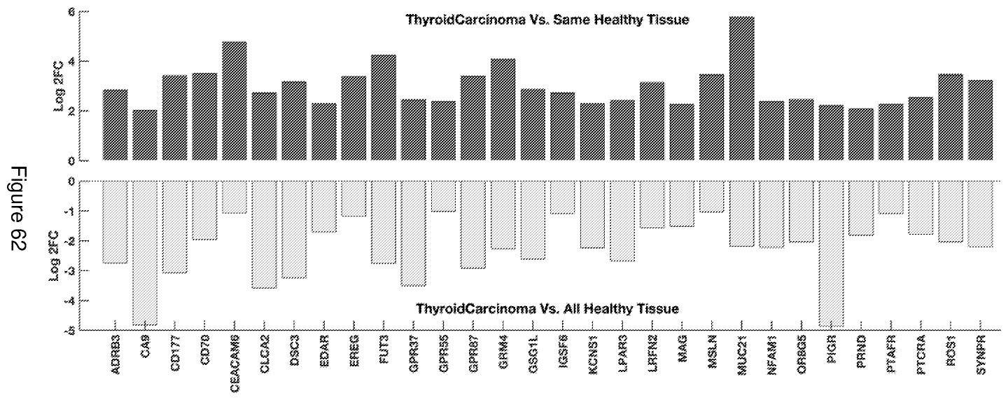

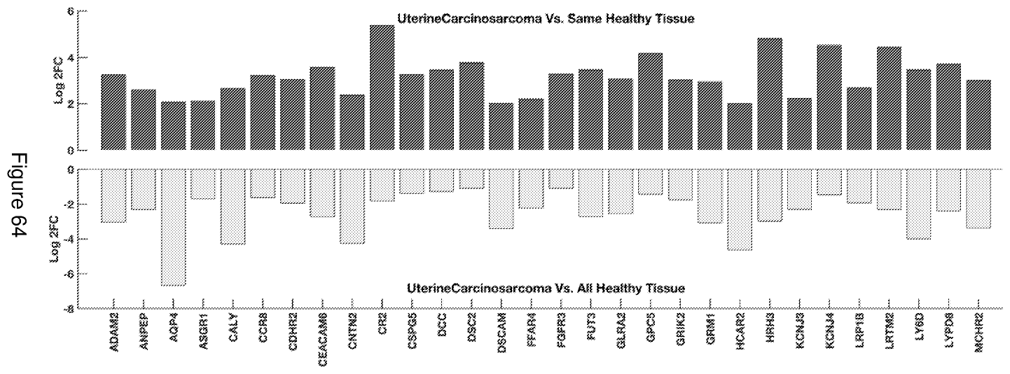

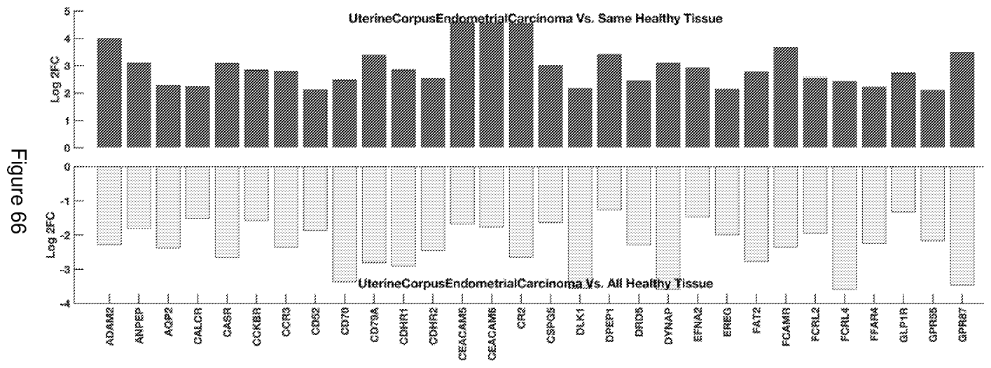

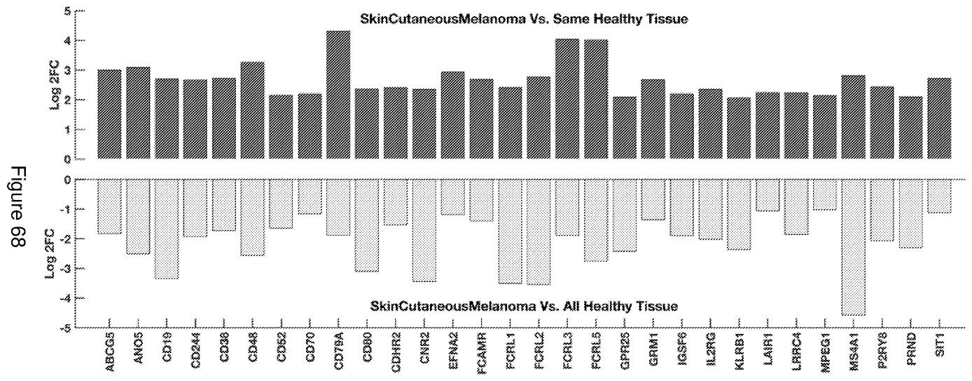

Anticancer drugs that target CSRs are also likely to exhibit off-target adverse effects that are related to the expression of CSRs in other healthy tissues. Therefore, the best drug target CSR would be those that are upregulated in the disease state compared to any other healthy body tissues. To identify such CSR targets, we performed differential gene expression using the negative binomial test (Anders and Huber (2010)) by comparing the CSR transcript levels between each cancer type from the TCGA and those of each of the healthy tissues from the GTEx project. The healthy tissues that were studied/screened by the inventors include adipose tissue, adrenal gland, bladder, blood, blood vessel, bone marrow, brain, breast, cervix, colon, esophagus, fallopian tube, heart, kidney, liver, lung, muscle, nerve, ovary, pancreas, pituitary, prostate, salivary gland, skin, small intestine, spleen, stomach, testis, thyroid, uterus, and vagina. The CSR targets which were identified for systemic targeting for each type of cancer which was screened are shown in Table 1 and in FIGS. 17, 19, 21, 23, 25, 27, 29, 31, 33, 35, 37, 39, 41, 43, 45, 47, 49, 51, 53, 55, 57, 59, 61, 63, 65 and 67. The inventors returned the CSR transcripts that were found to be upregulated (adjusted p-value<0.05 and log 2 fold change>2) on tumour cells in comparison to the healthy normal cells that the tumour cells originate from, as well as with upregulated (adjusted p-value<0.05 and log 2 fold change>2) on tumour cells in comparison healthy tissue comparisons to yield “ideal” drug and antibody targets. Anticancer agents targeted to these ideal targets can be administered systemically, since it is highly unlikely that off target effects will occur in the subject.

TABLE 1
List of ideal targets identified for the specified cancer entities

| Cancer | Ideal Targets Identified |
|---|---|
| Acute Myeloid | Cytokine receptor-like factor 2 (CRLF2) (UniProtKB - A0A0C4DH06); |
| Leukemia | Proepiregulin (EREG) (UniProtKB - O14944); Receptor-type tyrosine- |
| | protein kinase FLT3 (FLT3) (UniProtKB - E7ER61); Probable G-protein |
| | coupled receptor 32 (GPR32) (UniProtKB - H9NIL6); Mast/stem cell |
| | growth factor receptor Kit (KIT) (UniProtKB - A0A024RDA0); Olfactory |
| | receptor 1L4 (OR1L4) (UniProtKB - Q8NGR5); Olfactory receptor 1L6 |
| | (OR1L6) (UniProtKB - A0A0C4DFP2); Olfactory receptor 1N2 (OR1N2) |
| | (UniProtKB - A0A126GW94); Olfactory receptor 2G2 (OR2G2) (UniProtKB - |
| | Q8NGZ5); Olfactory receptor 2G3 (OR2G3) (UniProtKB - A0A126GVX0); |
| | Olfactory receptor 2L3 (OR2L3) (UniProtKB - Q8NG85); Olfactory receptor |
| | 2L5 (OR2L5) (UniProtKB - A0A126GWR8); Olfactory receptor 2L8 |
| | (OR2L8) (UniProtKB - Q8NGY9); Olfactory receptor 5C1 (OR5C1) |
| | (UniProtKB - A0A126GW42); Olfactory receptor 9A4 (OR9A4) (UniProtKB - |
| | A0A126GVB1); Placenta-expressed transcript 1 protein (PLET1) |
| | (UniProtKB - Q6UQ28); Relaxin receptor 2 (RXFP2) (UniProtKB - |
| | Q8WXD0); Protein spinster homolog 3 (SPNS3) (UniProtKB - I3L3W7); |
| Adrenocortical | Protein delta homolog 1 (DLK1) (UniProtKB - G3V2R7); Ectodysplasin-A |
| Carcinoma | (EDA) (UniProtKB - A0A0C4DGX3); and Pyroglutamylated RFamide |
| | peptide receptor (QRFPR) (UniProtKB - F2Z3L3). |
| Brain Lower Grade | Aquaporin-4 (AQP4) (UniProtKB - F1DSG4); Acid-sensing ion channel 1 |
| Glioma | (ASIC1) (UniProtKB - F8VSK4); Astrotactin-1 (ASTN1) (UniProtKB - |
| | B1AJS1); Astrotactin-2 (ASTN2) (UniProtKB - B7ZKP5); Cell adhesion |
| | molecule 2 (CADM2) (UniProtKB - G3XHN4); Contactin-1 (CNTN1) |
| | (UniProtKB - A0A024R104); Chondroitin sulfate proteoglycan 5 (CSPG5) |
| | (UniProtKB - A0A087WUT8); Epithelial discoidin domain-containing |
| | receptor 1 (DDR1) (UniProtKB - A0A024RCJ0); Delta-like protein 3 (DLL3) |
| | (UniProtKB - M0R177); Delta and Notch-like epidermal growth factor- |
| | related receptor (DNER) (UniProtKB - Q8NFT8); Down syndrome cell |
| | adhesion molecule (DSCAM) (UniProtKB - A0A087WUI7); Endothelin B |
| | receptor (EDNRB) (UniProtKB - A0A024R638); Ephrin type-B receptor 1 |
| | (EPHB1) (UniProtKB - C9J466); Protocadherin Fat 3 (FAT3) (UniProtKB - |
| | E9PQ73); Glycine receptor subunit alpha-3 (GLRA3) (UniProtKB - |
| | O75311); Neuronal membrane glycoprotein M6-b (GPM6B) (UniProtKB - |
| | A0A024RBV7); Uracil nucleotide/cysteinyl leukotriene receptor (GPR17) |
| | (UniProtKB - C9JWY5); Probable G-protein coupled receptor 34 (GPR34) |
| | (UniProtKB - Q3SAH0); G-protein coupled receptor 39 (GPR39) |
| | (UniProtKB - A0A087WTL7); Probable G-protein coupled receptor 45 |
| | (GPR45) (UniProtKB - B5B0C1); Probable G-protein coupled receptor 75 |
| | (GPR75) (UniProtKB - O95800); Glutamate receptor 4 (GRIA4) |
| | (UniProtKB - E9PJZ5); Glutamate receptor ionotropic, delta-1 (GRID1) |
| | (UniProtKB - G3V186); Glutamate receptor ionotropic, delta-2 (GRID2) |
| | (UniProtKB - A0A087X043); Glutamate receptor ionotropic, kainate 3 |
| | (GRIK3) (UniProtKB - Q13003); Glutamate receptor ionotropic, kainate 4 |
| | (GRIK4) (UniProtKB - Q16099); Junctional adhesion molecule B (JAM2) |
| | (UniProtKB - P57087); Leucine-rich repeats and immunoglobulin-like |
| | domains protein 1 (LRIG1) (UniProtKB - Q96JA1); Low-density lipoprotein |
| | receptor-related protein 1B (LRP1B) (UniProtKB - E7ERG8); Low-density |
| | lipoprotein receptor-related protein 4 (LRP4) (UniProtKB - E9PNJ5); |
| | Leucine-rich repeat neuronal protein 1 (LRRN1) (UniProtKB - Q6UXK5); |
| | Ly6/PLAUR domain-containing protein 1 (LYPD1) (UniProtKB - F8WD77); |
| | MAM domain-containing glycosylphosphatidylinositol anchor protein 2 |
| | (MDGA2) (UniProtKB - E9PMG9); Membrane frizzled-related protein |
| | (MFRP) (UniProtKB - A0A0U1RQG2); Matrix metalloproteinase-16 |
| | (MMP16) (UniProtKB - E5RJA7); Neural cell adhesion molecule 1 |
| | (NCAM1) (UniProtKB - A0A087WTE4); Neuroligin-1 (NLGN1) (UniProtKB - |
| | C9J4D3); Neuroligin-3 (NLGN3) (UniProtKB - A0A087WW27); Neuronal |
| | cell adhesion molecule (NRCAM) (UniProtKB - A0A087X2B3); BDNF/NT-3 |
| | growth factors receptor (NTRK2) (UniProtKB - A0A024R230); Olfactory |
| | receptor 4K2 (OR4K2) (UniProtKB - A0A126GVP5); P2X purinoceptor 7 |
| | (P2RX7) (UniProtKB - F5H237); RPE-retinal G protein-coupled receptor |
| | (RGR) (UniProtKB - A0A0S2Z494); Roundabout homolog 1 (ROBO1) |
| | (UniProtKB - A0A087WTM1); Sodium channel protein type 3 subunit alpha |
| | (SCN3A) (UniProtKB - A0A1W2PQ58); Triggering receptor expressed on |
| | myeloid cells 2 (TREM2) (UniProtKB - Q5TCX1); Protein tweety homolog 2 |
| | (TTYH2) (UniProtKB - B4DKD1); Vasoactive intestinal polypeptide |
| | receptor 2 (VIPR2) (UniProtKB - C9JCP7). |
| Breast Invasive | T-cell surface glycoprotein CD1b (CD1B) (UniProtKB - P29016); Claudin-6 |
| Carcinoma | (CLDN6) (UniProtKB - I3L1E7); Receptor tyrosine-protein kinase erbB-2 |
| | (ERBB2) (UniProtKB - B4DTR1); Receptor tyrosine-protein kinase erbB-3 |
| | (ERBB3) (UniProtKB - B3KWG5); N-formyl peptide receptor 3 (FPR3) |
| | (UniProtKB - P25089); GDNF family receptor alpha-1 (GFRA1) (UniProtKB - |
| | P56159); Glycine receptor subunit alpha-3 (GLRA3) (UniProtKB - |
| | O75311); Hydroxycarboxylic acid receptor 1 (HCAR1) (UniProtKB - |
| | Q9BXC0); Insulin-like growth factor 1 receptor (IGF1R) (UniProtKB - |
| | C9J5X1); Leucine-rich repeat-containing protein 15 (LRRC15) (UniProtKB - |
| | Q8TF66); Neuropeptide Y receptor type 1 (NPY1R) (UniProtKB - |
| | B4DKL9); Prolactin receptor (PRLR) (UniProtKB - D6R9E1); Receptor- |
| | type tyrosine-protein phosphatase T (PTPRT) (UniProtKB - A0A075B6H0); |
| | Proto-oncogene tyrosine-protein kinase receptor Ret (RET) (UniProtKB - |
| | A0A024R7T2); Zinc transporter ZIP6 (SLC39A6) (UniProtKB - K7EQ91); |
| | SLIT and NTRK-like protein 6 (SLITRK6) (UniProtKB - Q9H5Y7); |
| | Triggering receptor expressed on myeloid cells 2 (TREM2) (UniProtKB - |
| | Q5TCX1); V-set domain-containing T-cell activation inhibitor 1 (VTCN1) |
| | (UniProtKB - A0A087X1M4). |
| Cervical Squamous | Cadherin-3 (CDH3) (UniProtKB - J3QL75); Calcium-activated chloride |
| Cell Carcinoma And | channel regulator 2 (CLCA2) (UniProtKB - Q9UQC9); Receptor tyrosine- |
| Endocervical | protein kinase erbB-2 (ERBB2) (UniProtKB - B4DTR1); G-protein coupled |
| Adenocarcinoma | receptor 87 (GPR87) (UniProtKB - Q9BY21); Lymphocyte antigen 6K |
| | (LY6K) (UniProtKB - E5RGJ8); Mesothelin (MSLN) (UniProtKB - H3BR90); |
| | Mucin-16 (MUC16) (UniProtKB - Q8WXI7). |
| Cholangiocarcinoma | Kidney-associated antigen 1 (KAAG1) (UniProtKB - Q9UBP8); V-set |
| | domain-containing T-cell activation inhibitor 1 (VTCN1) (UniProtKB - |
| | A0A087X1M4). |
| Colon Adenocarcinoma | Carcinoembryonic antigen-related cell adhesion molecule 5 (CEACAM5) |
| | (UniProtKB - A0A024R0K5); Carcinoembryonic antigen-related cell |
| | adhesion molecule 6 (CEACAM6) (UniProtKB - M0QYD3); Claudin-4 |
| | (CLDN4) (UniProtKB - O14493); Tumor necrosis factor receptor |
| | superfamily member EDAR (EDAR) (UniProtKB - Q9UNE0); Ephrin type-B |
| | receptor 2 (EPHB2) (UniProtKB - B1AKC9); Metabotropic glutamate |
| | receptor 8 (GRM8) (UniProtKB - A0A0A0MT06); Neuropeptide S receptor |
| | (NPSR1) (UniProtKB - A0A090N8Z1); E3 ubiquitin-protein ligase RNF43 |
| | (RNF43) (UniProtKB - J3KSE3). |
| Esophageal Carcinoma | Anoctamin-1 (ANO1) (UniProtKB - E9PNA7); Amphiregulin (AREG) |
| | (UniProtKB - D6RFX5); Attractin (ATRN) (UniProtKB - O75882); Beta- |
| | secretase 2 (BACE2) (UniProtKB - Q9Y5Z0); Bone morphogenetic protein |
| | receptor type-2 (BMPR2) (UniProtKB - A0A1W2PPB5); T-cell surface |
| | glycoprotein CD1b (CD1B) (UniProtKB - P29016); CD276 antigen (CD276) |
| | (UniProtKB - A0A0C4DGH0); CD9 antigen (CD9) (UniProtKB - |
| | A0A087WU13); CUB domain-containing protein 1 (CDCP1) (UniProtKB - |
| | Q9H5V8); Cadherin-3 (CDH3) (UniProtKB - J3QL75); Carcinoembryonic |
| | antigen-related cell adhesion molecule 5 (CEACAM5) (UniProtKB - |
| | A0A024R0K5); Carcinoembryonic antigen-related cell adhesion molecule |
| | 6 (CEACAM6) (UniProtKB - M0QYD3); Calcium-activated chloride channel |
| | regulator 2 (CLCA2) (UniProtKB - Q9UQC9); Claudin-4 (CLDN4) |
| | (UniProtKB - O14493); Claudin-6 (CLDN6) (UniProtKB - I3L1E7); Epithelial |
| | discoidin domain-containing receptor 1 (DDR1) (UniProtKB - |
| | A0A024RCJ0); Desmocollin-2 (DSC2) (UniProtKB - Q02487); |
| | Desmocollin-3 (DSC3) (UniProtKB - Q14574); Desmoglein-2 (DSG2) |
| | (UniProtKB - J3KSI6); Desmoglein-3 (DSG3) (UniProtKB - P32926); |
| | Tumor necrosis factor receptor superfamily member EDAR (EDAR) |
| | (UniProtKB - Q9UNE0); Ephrin-A4 (EFNA4) (UniProtKB - P52798); Ephrin- |
| | B1 (EFNB1) (UniProtKB - P98172); Ephrin-B2 (EFNB2) (UniProtKB - |
| | P52799); Epidermal growth factor receptor (EGFR) (UniProtKB - |
| | A0A1W2PRR9); Epithelial cell adhesion molecule (EPCAM) (UniProtKB - |
| | B5MCA4); Ephrin type-A receptor 2 (EPHA2) (UniProtKB - A0A024QZA8); |
| | Ephrin type-B receptor 2 (EPHB2) (UniProtKB - B1AKC9); Ephrin type-B |
| | receptor 3 (EPHB3) (UniProtKB - P54753); Receptor tyrosine-protein |
| | kinase erbB-2 (ERBB2) (UniProtKB - B4DTR1); Receptor tyrosine-protein |
| | kinase erbB-3 (ERBB3) (UniProtKB - B3KWG5); Proepiregulin (EREG) |
| | (UniProtKB - O14944); Protocadherin Fat 1 (FAT1) (UniProtKB - |
| | A0A087WVP1); Protocadherin Fat 2 (FAT2) (UniProtKB - Q9NYQ8); |
| | FRAS1-related extracellular matrix protein 2 (FREM2) (UniProtKB - ); |
| | Frizzled-10 (FZD10) (UniProtKB - F5H450); Frizzled-6 (FZD6) (UniProtKB - |
| | A0A024R9E9); Frizzled-7 (FZD7) (UniProtKB - O75084); Glypican-1 |
| | (GPC1) (UniProtKB - C9J4Y6); Transmembrane glycoprotein NMB |
| | (GPNMB) (UniProtKB - A0A024RA55); Probable G-protein coupled |
| | receptor 33 (GPR33) (UniProtKB - Q49SQ1); G-protein coupled receptor |
| | 87 (GPR87) (UniProtKB - Q9BY21); Insulin-like growth factor 1 receptor |
| | (IGF1R) (UniProtKB - C9J5X1); Immunoglobulin superfamily member 3 |
| | (IGSF3) (UniProtKB - O75054); Immunoglobulin-like domain-containing |
| | receptor 1 (ILDR1) (UniProtKB - Q86SU0); Integrin alpha-V (ITGAV) |
| | (UniProtKB - L7RXH0); Lysosome-associated membrane glycoprotein 1 |
| | (LAMP1) (UniProtKB - A0A024RDY3); Lysophosphatidic acid receptor 3 |
| | (LPAR3) (UniProtKB - Q9UBY5); Leucine-rich repeat and fibronectin type- |
| | III domain-containing protein 3 (LRFN3) (UniProtKB - Q9BTN0); Low- |
| | density lipoprotein receptor-related protein 11 (LRP11) (UniProtKB - |
| | Q5VYB9); Low-density lipoprotein receptor-related protein 5 (LRP5) |
| | (UniProtKB - E9PHY1); Leucine-rich repeat-containing protein 15 |
| | (LRRC15) (UniProtKB - Q8TF66); Lymphocyte antigen 6E (LY6E) |
| | (UniProtKB - E5RGI6); Hepatocyte growth factor receptor (MET) |
| | (UniProtKB - A0A024R728); Mesothelin (MSLN) (UniProtKB - H3BR90); |
| | Macrophage-stimulating protein receptor (MST1R) (UniProtKB - |
| | A0A087WZQ8); Mucin-17 (MUC17) (UniProtKB - E7EPM4); Mucin-4 |
| | (MUC4) (UniProtKB - A0A0G2JLU8); Neogenin (NEO1) (UniProtKB - |
| | Q59FP8); Neuromedin-U receptor 2 (NMUR2) (UniProtKB - Q9GZQ4); |
| | Neuropeptide S receptor (NPSR1) (UniProtKB - A0A090N8Z1); Pro- |
| | neuregulin-1, membrane-bound isoform (NRG1) (UniProtKB - |
| | A0A087X0X9); Olfactory receptor 1L6 (OR1L6) (UniProtKB - |
| | A0A0C4DFP2); Olfactory receptor 1N2 (OR1N2) (UniProtKB - |
| | A0A126GW94); Olfactory receptor 2A4 (OR2A4) (UniProtKB - |
| | A0A126GVW2); Olfactory receptor 2A7 (OR2A7) (UniProtKB - |
| | A0A126GWD8); Olfactory receptor 4K2 (OR4K2) (UniProtKB - |
| | A0A126GVP5); Olfactory receptor 5C1 (OR5C1) (UniProtKB - |
| | A0A126GW42); Olfactory receptor 9A4 (OR9A4) (UniProtKB - |
| | A0A126GVB1); Protocadherin-1 (PCDH1) (UniProtKB - D6RAX3); GPI |
| | ethanolamine phosphate transferase 3 (PIGO) (UniProtKB - Q8TEQ8); |
| | GPI transamidase component PIG-T (PIGT) (UniProtKB - A0A1W2PNP0); |
| | Prominin-2 (PROM2) (UniProtKB - F8WDW5); Receptor-type tyrosine- |
| | protein phosphatase F (PTPRF) (UniProtKB - A2A437); Receptor-type |
| | tyrosine-protein phosphatase kappa (PTPRK) (UniProtKB - E9PGC5); |
| | Pyroglutamylated RFamide peptide receptor (QRFPR) (UniProtKB - |
| | F2Z3L3); RELT-like protein 1 (RELL1) (UniProtKB - D6RBN9); E3 |
| | ubiquitin-protein ligase RNF43 (RNF43) (UniProtKB - J3KSE3); Zinc |
| | transporter ZIP6 (SLC39A6) (UniProtKB - K7EQ91); Choline transporter- |
| | like protein 4 (SLC44A4) (UniProtKB - A0A0G2JL76); Trace amine- |
| | associated receptor 5 (TAAR5) (UniProtKB - O14804); Protransforming |
| | growth factor alpha (TGFA) (UniProtKB - E7EPT6); Toll-like receptor 3 |
| | (TLR3) (UniProtKB - D6RA51); Transmembrane 4 L6 family member 1 |
| | (TM4SF1) (UniProtKB - C9J611); Transmembrane channel-like protein 7 |
| | (TMC7) (UniProtKB - H3BNW8); Triggering receptor expressed on myeloid |
| | cells 2 (TREM2) (UniProtKB - Q5TCX1); Protein tweety homolog 3 |
| | (TTYH3) (UniProtKB - A0A024R816); Netrin receptor UNC5B (UNC5B) |
| | (UniProtKB - Q8IZJ1). |
| Head And Neck | Amphiregulin (AREG) (UniProtKB - D6RFX5); T-cell surface glycoprotein |
| Squamous Cell | CD1b (CD1B) (UniProtKB - P29016); Cadherin-3 (CDH3) (UniProtKB - |
| Carcinoma | J3QL75); Calcium-activated chloride channel regulator 2 (CLCA2) |
| | (UniProtKB - Q9UQC9); Ephrin-B1 (EFNB1) (UniProtKB - P98172); |
| | Proepiregulin (EREG) (UniProtKB - O14944); G-protein coupled receptor |
| | 87 (GPR87) (UniProtKB - Q9BY21); Lysophosphatidic acid receptor 3 |
| | (LPAR3) (UniProtKB - Q9UBY5); Leucine-rich repeat-containing protein 15 |
| | (LRRC15) (UniProtKB - Q8TF66); Neuropeptide S receptor (NPSR1) |
| | (UniProtKB - A0A090N8Z1); Pro-neuregulin-1, membrane-bound isoform |
| | (NRG1) (UniProtKB - A0A087X0X9); Triggering receptor expressed on |
| | myeloid cells 2 (TREM2) (UniProtKB - Q5TCX1). |
| Kidney Chromophobe | Amyloid-like protein 2 (APLP2) (UniProtKB - E9PK76); Astrotactin-2 |
| | (ASTN2) (UniProtKB - B7ZKP5); Beta-secretase 2 (BACE2) (UniProtKB - |
| | Q9Y5Z0); Basigin (BSG) (UniProtKB - A0A087WUV8); CD9 antigen (CD9) |
| | (UniProtKB - A0A087WU13); Epithelial discoidin domain-containing |
| | receptor 1 (DDR1) (UniProtKB - A0A024RCJ0); Immunoglobulin-like |
| | domain-containing receptor 1 (ILDR1) (UniProtKB - Q86SU0); Mast/stem |
| | cell growth factor receptor Kit (KIT) (UniProtKB - A0A024RDA0); Leucine- |
| | rich repeat, immunoglobulin-like domain and transmembrane domain- |
| | containing protein 3 (LRIT3) (UniProtKB - A0A0A0MR64); Leucine-rich |
| | repeat neuronal protein 2 (LRRN2) (UniProtKB - A0A024R993); |
| | Hepatocyte growth factor receptor (MET) (UniProtKB - A0A024R728); |
| | Olfactory receptor 1L8 (OR1L8) (UniProtKB - A0A126GVC5); Olfactory |
| | receptor 1N1 (OR1N1) (UniProtKB - A0A087WXB1); 2-oxoglutarate |
| | receptor 1 (OXGR1) (UniProtKB - F5H3P1); Protocadherin-1 (PCDH1) |
| | (UniProtKB - D6RAX3); Putative transporter SVOPL (SVOPL) (UniProtKB - |
| | C9JVJ9). |
| Kidney Renal Clear | Calcitonin receptor (CALCR) (UniProtKB - A0A0A0MRG0); Cadherin-6 |
| Cell Carcinoma | (CDH6) (UniProtKB - D6RF86); Delta-like protein 4 (DLL4) (UniProtKB - |
| | Q9NR61); Dipeptidyl peptidase 4 (DPP4) (UniProtKB - F8WBB6); Ephrin- |
| | B2 (EFNB2) (UniProtKB - P52799); Ectonucleotide |
| | pyrophosphatase/phosphodiesterase family member 3 (ENPP3) |
| | (UniProtKB - E7ETI7); N-formyl peptide receptor 3 (FPR3) (UniProtKB - |
| | P25089); Probable G-protein coupled receptor 33 (GPR33) (UniProtKB - |
| | Q49SQ1); Metabotropic glutamate receptor 8 (GRM8) (UniProtKB - |
| | A0A0A0MT06); Vascular endothelial growth factor receptor 2 (KDR) |
| | (UniProtKB - A0A024RD88); Low-density lipoprotein receptor-related |
| | protein 2 (LRP2) (UniProtKB - E9PC35); Melanin-concentrating hormone |
| | receptor 1 (MCHR1) (UniProtKB - A6ZJ87); Hepatocyte growth factor |
| | receptor (MET) (UniProtKB - A0A024R728); Neuroligin-1 (NLGN1) |
| | (UniProtKB - C9J4D3); Neuropilin-1 (NRP1) (UniProtKB - E7EX60); |
| | Pyroglutamylated RFamide peptide receptor (QRFPR) (UniProtKB - |
| | F2Z3L3); Protransforming growth factor alpha (TGFA) (UniProtKB - |
| | E7EPT6); Toll-like receptor 3 (TLR3) (UniProtKB - D6RA51); Triggering |
| | receptor expressed on myeloid cells 2 (TREM2) (UniProtKB - Q5TCX1); |
| | Protein tweety homolog 3 (TTYH3) (UniProtKB - A0A024R816); Vascular |
| | cell adhesion protein 1 (VCAM1) (UniProtKB - E9PDD2). |
| Kidney Renal Papillary | Cadherin-6 (CDH6) (UniProtKB - D6RF86); Claudin-4 (CLDN4) |
| Cell Carcinoma | (UniProtKB - O14493); Contactin-6 (CNTN6) (UniProtKB - A0A024R2C7); |
| | Dipeptidyl peptidase 4 (DPP4) (UniProtKB - F8WBB6); High affinity |
| | immunoglobulin alpha and immunoglobulin mu Fc receptor (FCAMR) |
| | (UniProtKB - A0A0B4J1S2); Leucine-rich repeat transmembrane protein |
| | FLRT3 (FLRT3) (UniProtKB - Q9NZU0); Frizzled-1 (FZD1) (UniProtKB - |
| | Q9UP38); Immunoglobulin-like domain-containing receptor 1 (ILDR1) |
| | (UniProtKB - Q86SU0); Low-density lipoprotein receptor-related protein 2 |
| | (LRP2) (UniProtKB - E9PC35); Leucine-rich repeat neuronal protein 4 |
| | (LRRN4) (UniProtKB - Q8WUT4); Hepatocyte growth factor receptor |
| | (MET) (UniProtKB - A0A024R728); Pyroglutamylated RFamide peptide |
| | receptor (QRFPR) (UniProtKB - F2Z3L3); Trace amine-associated |
| | receptor 9 (TAAR9) (UniProtKB - Q96RI9); Triggering receptor expressed |
| | on myeloid cells 2 (TREM2) (UniProtKB - Q5TCX1); Zona pellucida-like |
| | domain-containing protein 1 (ZPLD1) (UniProtKB - Q8TCW7). |
| Liver Hepatocellular | Alpha-fetoprotein (AFP) (UniProtKB - J3KMX3); Glypican-3 (GPC3) |
| Carcinoma | (UniProtKB - I6QTG3). |
| Lung Adenocarcinoma | T-cell surface glycoprotein CD1b (CD1B) (UniProtKB - P29016); T-cell |
| | surface glycoprotein CD1e, membrane-associated (CD1E) (UniProtKB - |
| | P15812); Carcinoembryonic antigen-related cell adhesion molecule 6 |
| | (CEACAM6) (UniProtKB - M0QYD3); Claudin-6 (CLDN6) (UniProtKB - |
| | I3L1E7); Folate receptor alpha (FOLR1) (UniProtKB - A0A024R5H1); |
| | Leucine-rich repeat neuronal protein 4 (LRRN4) (UniProtKB - Q8WUT4); |
| | Hepatocyte growth factor receptor (MET) (UniProtKB - A0A024R728); |
| | Mesothelin (MSLN) (UniProtKB - H3BR90); Neuropeptide S receptor |
| | (NPSR1) (UniProtKB - A0A090N8Z1); Olfactory receptor 4C6 (OR4C6) |
| | (UniProtKB - A0A126GVN0); Sodium-dependent phosphate transport |
| | protein 2B (SLC34A2) (UniProtKB - D6RA94); Triggering receptor |
| | expressed on myeloid cells 2 (TREM2) (UniProtKB - Q5TCX1). |
| Lung Squamous Cell | Retinal-specific ATP-binding cassette transporter (ABCA4) (UniProtKB - |
| Carcinoma | F6TT59); CD9 antigen (CD9) (UniProtKB - A0A087WU13); Cadherin-3 |
| | (CDH3) (UniProtKB - J3QL75); Calcium-activated chloride channel |
| | regulator 2 (CLCA2) (UniProtKB - Q9UQC9); Claudin-6 (CLDN6) |
| | (UniProtKB - I3L1E7); Frizzled-6 (FZD6) (UniProtKB - A0A024R9E9); |
| | Transmembrane glycoprotein NMB (GPNMB) (UniProtKB - A0A024RA55); |
| | G-protein coupled receptor 87 (GPR87) (UniProtKB - Q9BY21); |
| | Neuropeptide S receptor (NPSR1) (UniProtKB - A0A090N8Z1); |
| | Transmembrane 4 L6 family member 1 (TM4SF1) (UniProtKB - C9J611); |
| | Triggering receptor expressed on myeloid cells 2 (TREM2) (UniProtKB - |
| | Q5TCX1); Uroplakin-1b (UPK1B) (UniProtKB - C9J027). |
| Lymphoid Neoplasm | C-C chemokine receptor type 5 (CCR5) (UniProtKB - P51681); CD180 |
| Diffuse Large B-cell | antigen (CD180) (UniProtKB - Q99467); T-cell surface glycoprotein CD1b |
| Lymphoma | (CD1B) (UniProtKB - P29016); B-cell antigen receptor complex-associated |
| | protein alpha chain (CD79A) (UniProtKB - M0QX61); B-cell antigen |
| | receptor complex-associated protein beta chain (CD79B) (UniProtKB - |
| | P40259); CD83 antigen (CD83) (UniProtKB - A0A087WX61); Cytotoxic T- |
| | lymphocyte protein 4 (CTLA4) (UniProtKB - P16410); C-X-C chemokine |
| | receptor type 5 (CXCR5) (UniProtKB - A0N0R2); Fc receptor-like protein 3 |
| | (FCRL3) (UniProtKB - Q96P31); 12-(S)-hydroxy-5,8,10,14- |
| | eicosatetraenoic acid receptor (GPR31) (UniProtKB - O00270); Probable |
| | G-protein coupled receptor 33 (GPR33) (UniProtKB - Q49SQ1); Probable |
| | G-protein coupled receptor 82 (GPR82) (UniProtKB - Q96P67); |
| | Interleukin-2 receptor subunit alpha (IL2RA) (UniProtKB - P01589); |
| | Lymphocyte activation gene 3 protein (LAG3) (UniProtKB - P18627); |
| | Sphingosine 1-phosphate receptor 2 (S1PR2) (UniProtKB - A0A024R7B2); |
| | T-cell immunoreceptor with Ig and ITIM domains (TIGIT) (UniProtKB - |
| | A0A0C4DGA4). |
| Mesothelioma | Ephrin-A5 (EFNA5) (UniProtKB - A0A087X240); Ephrin type-B receptor 2 |
| | (EPHB2) (UniProtKB - B1AKC9); Leucine-rich repeat transmembrane |
| | protein FLRT3 (FLRT3) (UniProtKB - Q9NZU0); Protein HEG homolog 1 |
| | (HEG1) (UniProtKB - A0A2R8Y4Z1); Intelectin-1 (ITLN1) (UniProtKB - |
| | Q8WWWA0); Leucine-rich repeat neuronal protein 4 (LRRN4) (UniProtKB - |
| | Q8WUT4); Lymphocyte antigen 6E (LY6E) (UniProtKB - E5RGI6); |
| | Hepatocyte growth factor receptor (MET) (UniProtKB - A0A024R728); |
| | Mesothelin (MSLN) (UniProtKB - H3BR90); Transmembrane 4 L6 family |
| | member 1 (TM4SF1) (UniProtKB - C9J611); Triggering receptor expressed |
| | on myeloid cells 2 (TREM2) (UniProtKB - Q5TCX1); Uroplakin-1b (UPK1B) |
| | (UniProtKB - C9J027); Uroplakin-3b (UPK3B) (UniProtKB - A0A0J9YVS6). |
| Ovarian Serous | Beta-secretase 2 (BACE2) (UniProtKB - Q9Y5Z0); Basal cell adhesion |
| Cystadenocarcinoma | molecule (BCAM) (UniProtKB - A0A087WXM8); CD276 antigen (CD276) |
| | (UniProtKB - A0A0C4DGH0); Leukocyte surface antigen CD47 (CD47) |
| | (UniProtKB - A0A2R8Y484); CD9 antigen (CD9) (UniProtKB - |
| | A0A087WU13); Cadherin-6 (CDH6) (UniProtKB - D6RF86); Claudin-4 |
| | (CLDN4) (UniProtKB - O14493); Claudin-6 (CLDN6) (UniProtKB - I3L1E7); |
| | Epithelial discoidin domain-containing receptor 1 (DDR1) (UniProtKB - |
| | A0A024RCJ0); D(4) dopamine receptor (DRD4) (UniProtKB - |
| | A0A0G2JM26); Ephrin-A4 (EFNA4) (UniProtKB - P52798); Ephrin-B2 |
| | (EFNB2) (UniProtKB - P52799); Ephrin type-B receptor 2 (EPHB2) |
| | (UniProtKB - B1AKC9); Receptor tyrosine-protein kinase erbB-2 (ERBB2) |
| | (UniProtKB - B4DTR1); Folate receptor alpha (FOLR1) (UniProtKB - |
| | A0A024R5H1); N-formyl peptide receptor 3 (FPR3) (UniProtKB - P25089); |
| | Frizzled-10 (FZD10) (UniProtKB - F5H450); Frizzled-2 (FZD2) (UniProtKB - |
| | Q14332); Frizzled-3 (FZD3) (UniProtKB - E5RGI9); Frizzled-6 (FZD6) |
| | (UniProtKB - A0A024R9E9); 12-(S)-hydroxy-5,8,10,14-eicosatetraenoic |
| | acid receptor (GPR31) (UniProtKB - O00270); G-protein coupled receptor |
| | 39 (GPR39) (UniProtKB - A0A087WTL7); Immunoglobulin-like domain- |
| | containing receptor 1 (ILDR1) (UniProtKB - Q86SU0); Interphotoreceptor |
| | matrix proteoglycan 2 (IMPG2) (UniProtKB - F1T0J3); Lysophosphatidic |
| | acid receptor 3 (LPAR3) (UniProtKB - Q9UBY5); Leucine-rich repeat and |
| | fibronectin type-III domain-containing protein 3 (LRFN3) (UniProtKB - |
| | Q9BTN0); Leucine-rich repeat neuronal protein 2 (LRRN2) (UniProtKB - |
| | A0A024R993); Lymphocyte antigen 6E (LY6E) (UniProtKB - E5RGI6); |
| | Ly6/PLAUR domain-containing protein 1 (LYPD1) (UniProtKB - F8WD77); |
| | Melanocortin receptor 4 (MC4R) (UniProtKB - P32245); Mesothelin |
| | (MSLN) (UniProtKB - H3BR90); Mucin-16 (MUC16) (UniProtKB - |
| | Q8WXI7); Neogenin (NEO1) (UniProtKB - Q59FP8); Olfactory receptor |
| | 1M1 (OR1M1) (UniProtKB - Q8NGA1); Olfactory receptor 1N1 (OR1N1) |
| | (UniProtKB - A0A087WXB1); Otopetrin-1 (OTOP1) (UniProtKB - |
| | Q7RTM1); 2-oxoglutarate receptor 1 (OXGR1) (UniProtKB - F5H3P1); |
| | Protocadherin-1 (PCDH1) (UniProtKB - D6RAX3); GPI ethanolamine |
| | phosphate transferase 3 (PIGO) (UniProtKB - Q8TEQ8); GPI |
| | transamidase component PIG-T (PIGT) (UniProtKB - A0A1W2PNP0); |
| | Parathyroid hormone 2 receptor (PTH2R) (UniProtKB - B4DFN8); |
| | Receptor-type tyrosine-protein phosphatase F (PTPRF) (UniProtKB - |
| | A2A437); E3 ubiquitin-protein ligase RNF43 (RNF43) (UniProtKB - |
| | J3KSE3); Sodium-dependent phosphate transport protein 2B (SLC34A2) |
| | (UniProtKB - D6RA94); Putative transporter SVOPL (SVOPL) (UniProtKB - |
| | C9JVJ9); Toll-like receptor 3 (TLR3) (UniProtKB - D6RA51); |
| | Transmembrane 4 L6 family member 1 (TM4SF1) (UniProtKB - C9J611); |
| | Trophoblast glycoprotein-like (TPBGL) (UniProtKB - P0DKB5); Triggering |
| | receptor expressed on myeloid cells 2 (TREM2) (UniProtKB - Q5TCX1); |
| | Protein tweety homolog 3 (TTYH3) (UniProtKB - A0A024R816); Netrin |
| | receptor UNC5B (UNC5B) (UniProtKB - Q8IZJ1); Uroplakin-3b (UPK3B) |
| | (UniProtKB - A0A0J9YVS6); V-set domain-containing T-cell activation |
| | inhibitor 1 (VTCN1) (UniProtKB - A0A087X1M4); Zona pellucida sperm- |
| | binding protein 3 (ZP3) (UniProtKB - E9PFI9). |
| Pancreatic | Carcinoembryonic antigen-related cell adhesion molecule 6 (CEACAM6) |
| Adenocarcinoma | (UniProtKB - M0QYD3); Mesothelin (MSLN) (UniProtKB - H3BR90); |
| | Neuropeptide S receptor (NPSR1) (UniProtKB - A0A090N8Z1); Triggering |
| | receptor expressed on myeloid cells 2 (TREM2) (UniProtKB - Q5TCX1). |
| Prostate | Astrotactin-2 (ASTN2) (UniProtKB - B7ZKP5); Basal cell adhesion |
| Adenocarcinoma | molecule (BCAM) (UniProtKB - A0A087WXM8); Dipeptidyl peptidase 4 |
| | (DPP4) (UniProtKB - F8WBB6); Ectonucleotide |
| | pyrophosphatase/phosphodiesterase family member 5 (ENPP5) |
| | (UniProtKB - Q9UJA9); Glutamate carboxypeptidase 2 (FOLH1) |
| | (UniProtKB - E9PI29); Interleukin-5 receptor subunit alpha (IL5RA) |
| | (UniProtKB - A0A024R2C8); Immunoglobulin-like domain-containing |
| | receptor 1 (ILDR1) (UniProtKB - Q86SU0); Lysophosphatidic acid receptor |
| | 3 (LPAR3) (UniProtKB - Q9UBY5); Low-density lipoprotein receptor- |
| | related protein 11 (LRP11) (UniProtKB - Q5VYB9); Leucine-rich repeat |
| | neuronal protein 1 (LRRN1) (UniProtKB - Q6UXK5); Neuropeptide Y |
| | receptor type 4 (NPY4R) (UniProtKB - P50391); Zinc transporter ZIP6 |
| | (SLC39A6) (UniProtKB - K7EQ91); Choline transporter-like protein 4 |
| | (SLC44A4) (UniProtKB - A0A0G2JL76); Metalloreductase STEAP1 |
| | (STEAP1) (UniProtKB - Q9UHE8); Tomoregulin-2 (TMEFF2) (UniProtKB - |
| | Q9UIK5). |
| Skin Cutaneous | ATP-binding cassette sub-family B member 5 (ABCB5) (UniProtKB - |
| Melanoma | H7C165); Beta-secretase 2 (BACE2) (UniProtKB - Q9Y5Z0); CD276 |
| | antigen (CD276) (UniProtKB - A0A0C4DGH0); CD63 antigen (CD63) |
| | (UniProtKB - A0A024RB05); Delta-like protein 3 (DLL3) (UniProtKB - |
| | M0R177); Endothelin B receptor (EDNRB) (UniProtKB - A0A024R638); |
| | Receptor tyrosine-protein kinase erbB-3 (ERBB3) (UniProtKB - B3KWG5); |
| | Transmembrane glycoprotein NMB (GPNMB) (UniProtKB - A0A024RA55); |
| | Immunoglobulin superfamily member 3 (IGSF3) (UniProtKB - O75054); |
| | Mast/stem cell growth factor receptor Kit (KIT) (UniProtKB - |
| | A0A024RDA0); Lymphocyte antigen 6E (LY6E) (UniProtKB - E5RGI6); |
| | Olfactory receptor 5J2 (OR5J2) (UniProtKB - A0A126GVP0); Olfactory |
| | receptor 7A5 (OR7A5) (UniProtKB - A0A126GW60); Olfactory receptor |
| | 9G1 (OR9G1) (UniProtKB - Q8NH87); P2X purinoceptor 7 (P2RX7) |
| | (UniProtKB - F5H237); Melanocyte protein PMEL (PMEL) (UniProtKB - |
| | F8VUB1); Zinc transporter ZIP6 (SLC39A6) (UniProtKB - K7EQ91); |
| | Protein tweety homolog 3 (TTYH3) (UniProtKB - A0A024R816); 5,6- |
| | dihydroxyindole-2-carboxylic acid oxidase (TYRP1) (UniProtKB - C9JZ52). |
| Stomach | Beta-secretase 2 (BACE2) (UniProtKB - Q9Y5Z0); Signal transducer |
| Adenocarcinoma | CD24 (CD24) (UniProtKB - A0A087WU21); Carcinoembryonic antigen- |
| | related cell adhesion molecule 5 (CEACAM5) (UniProtKB - A0A024R0K5); |
| | Carcinoembryonic antigen-related cell adhesion molecule 6 (CEACAM6) |
| | (UniProtKB - M0QYD3); Claudin-4 (CLDN4) (UniProtKB - O14493); |
| | Claudin-6 (CLDN6) (UniProtKB - I3L1E7); Epithelial discoidin domain- |
| | containing receptor 1 (DDR1) (UniProtKB - A0A024RCJ0); Tumor necrosis |
| | factor receptor superfamily member EDAR (EDAR) (UniProtKB - |
| | Q9UNE0); Ephrin-A2 (EFNA2) (UniProtKB - O43921); Ephrin-B2 (EFNB2) |
| | (UniProtKB - P52799); Epithelial cell adhesion molecule (EPCAM) |
| | (UniProtKB - B5MCA4); Ephrin type-B receptor 2 (EPHB2) (UniProtKB - |
| | B1AKC9); Receptor tyrosine-protein kinase erbB-2 (ERBB2) (UniProtKB - |
| | B4DTR1); Receptor tyrosine-protein kinase erbB-3 (ERBB3) (UniProtKB - |
| | B3KWG5); Protocadherin Fat 1 (FAT1) (UniProtKB - A0A087WVP1); N- |
| | formyl peptide receptor 3 (FPR3) (UniProtKB - P25089); Probable G- |
| | protein coupled receptor 25 (GPR25) (UniProtKB - O00155); Probable G- |
| | protein coupled receptor 33 (GPR33) (UniProtKB - Q49SQ1); G-protein |
| | coupled receptor 35 (GPR35) (UniProtKB - Q9HC97); Immunoglobulin-like |
| | domain-containing receptor 1 (ILDR1) (UniProtKB - Q86SU0); Low-density |
| | lipoprotein receptor-related protein 11 (LRP11) (UniProtKB - Q5VYB9); |
| | Lymphocyte antigen 6E (LY6E) (UniProtKB - E5RGI6); Hepatocyte growth |
| | factor receptor (MET) (UniProtKB - A0A024R728); Mesothelin (MSLN) |
| | (UniProtKB - H3BR90); Mucin-13 (MUC13) (UniProtKB - C9IZG1); |
| | Neuromedin-U receptor 2 (NMUR2) (UniProtKB - Q9GZQ4); Neuropeptide |
| | S receptor (NPSR1) (UniProtKB - A0A090N8Z1); Olfactory receptor 1N2 |
| | (OR1N2) (UniProtKB - A0A126GW94); Olfactory receptor 4C6 (OR4C6) |
| | (UniProtKB - A0A126GVN0); Olfactory receptor 4K2 (OR4K2) (UniProtKB - |
| | A0A126GVP5); Olfactory receptor 5C1 (OR5C1) (UniProtKB - |
| | A0A126GW42); Olfactory receptor 6C6 (OR6C6) (UniProtKB - |
| | A0A126GW15); Olfactory receptor 6Y1 (OR6Y1) (UniProtKB - Q8NGX8); |
| | Olfactory receptor 7C2 (OR7C2) (UniProtKB - O60412); Olfactory receptor |
| | 7G1 (OR7G1) (UniProtKB - A0A126GVS6); Olfactory receptor 7G3 |
| | (OR7G3) (UniProtKB - A0A126GVR4); Protocadherin-1 (PCDH1) |
| | (UniProtKB - D6RAX3); GPI transamidase component PIG-T (PIGT) |
| | (UniProtKB - A0A1W2PNP0); Prostasin (PRSS8) (UniProtKB - H3BUJ8); |
| | Receptor-type tyrosine-protein phosphatase kappa (PTPRK) (UniProtKB - |
| | E9PGC5); RELT-like protein 1 (RELL1) (UniProtKB - D6RBN9); E3 |
| | ubiquitin-protein ligase RNF43 (RNF43) (UniProtKB - J3KSE3); Choline |
| | transporter-like protein 4 (SLC44A4) (UniProtKB - A0A0G2JL76); Trace |
| | amine-associated receptor 1 (TAAR1) (UniProtKB - Q96RJ0); Trace |
| | amine-associated receptor 5 (TAAR5) (UniProtKB - O14804); Toll-like |
| | receptor 3 (TLR3) (UniProtKB - D6RA51); Transmembrane 4 L6 family |
| | member 1 (TM4SF1) (UniProtKB - C9J611); Triggering receptor expressed |
| | on myeloid cells 2 (TREM2) (UniProtKB - Q5TCX1); Protein tweety |
| | homolog 3 (TTYH3) (UniProtKB - A0A024R816). |
| Uterine | Claudin-6 (CLDN6) (UniProtKB - I3L1E7); Ephrin-A4 (EFNA4) (UniProtKB - |
| Carcinosarcoma | P52798); Ephrin type-B receptor 2 (EPHB2) (UniProtKB - B1AKC9); |
| | Frizzled-2 (FZD2) (UniProtKB - Q14332); Placenta-specific protein 1 |
| | (PLAC1) (UniProtKB - Q9HBJ0); Protein tweety homolog 3 (TTYH3) |
| | (UniProtKB - A0A024R816). |
| Uterine Corpus | Claudin-6 (CLDN6) (UniProtKB - I3L1E7); V-set domain-containing T-cell |
| Endometrial Carcinoma | activation inhibitor 1 (VTCN1) (UniProtKB - A0A087X1M4). |
| Uveal Melanoma | ATP-binding cassette sub-family B member 5 (ABCB5) (UniProtKB - |
| | H7C165); CD44 antigen (CD44) (UniProtKB - A0A1W2PR55); CD63 |
| | antigen (CD63) (UniProtKB - A0A024RB05); Endothelin B receptor |
| | (EDNRB) (UniProtKB - A0A024R638); Protein ELFN1 (ELFN1) (UniProtKB - |
| | P0CZU0); Mast/stem cell growth factor receptor Kit (KIT) (UniProtKB - |
| | A0A024RDA0); Hepatocyte growth factor receptor (MET) (UniProtKB - |
| | A0A024R728); P2X purinoceptor 7 (P2RX7) (UniProtKB - F5H237); |
| | Melanocyte protein PMEL (PMEL) (UniProtKB - F8VUB1); Protein tweety |
| | homolog 3 (TTYH3) (UniProtKB - A0A024R816); 5,6-dihydroxyindole-2- |
| | carboxylic acid oxidase (TYRP1) (UniProtKB - C9JZ52). |

### Identification of the Other Targets

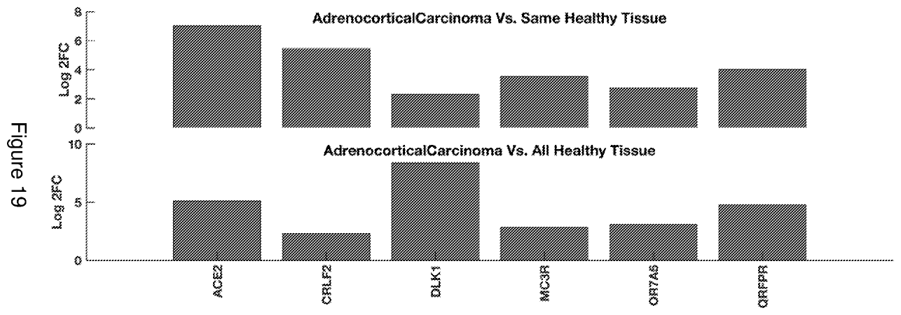

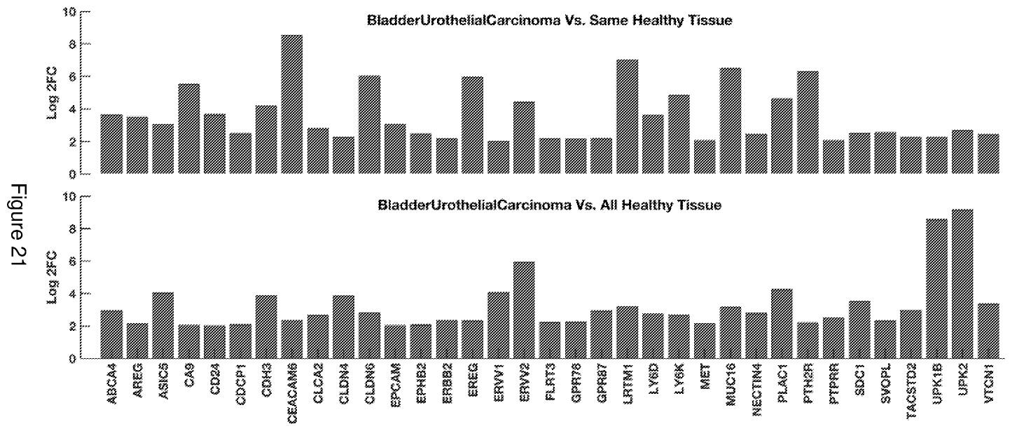

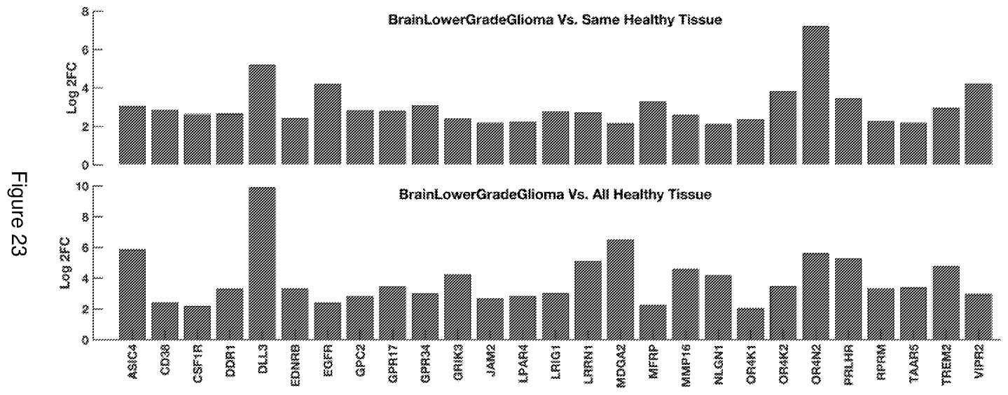

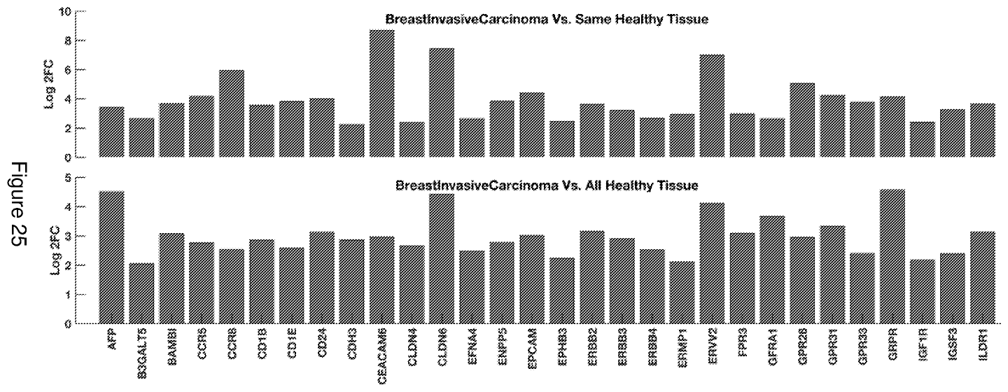

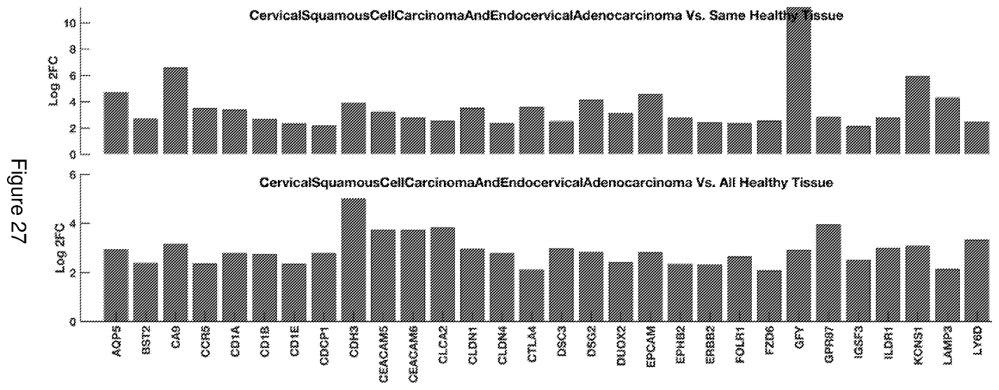

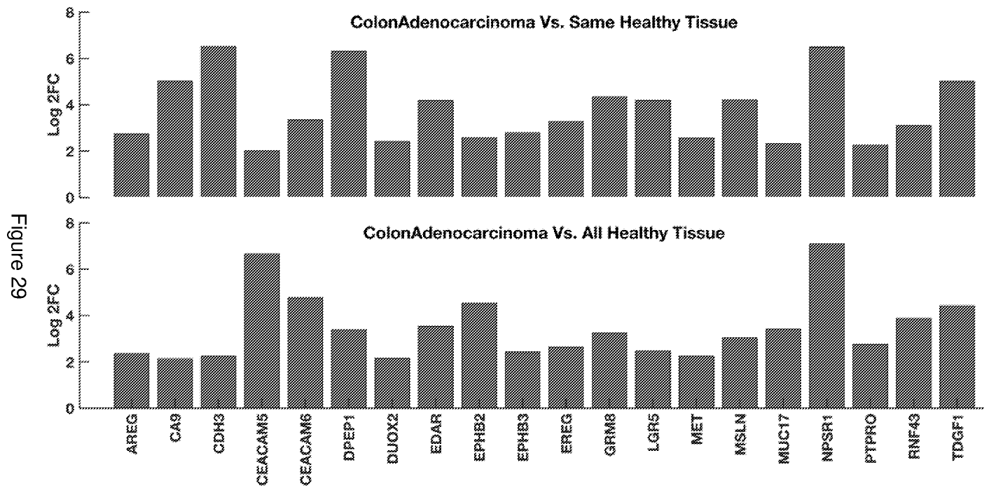

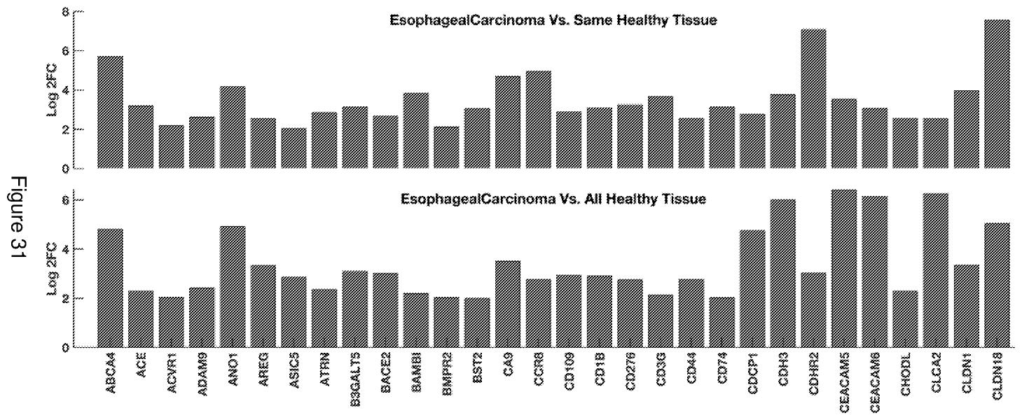

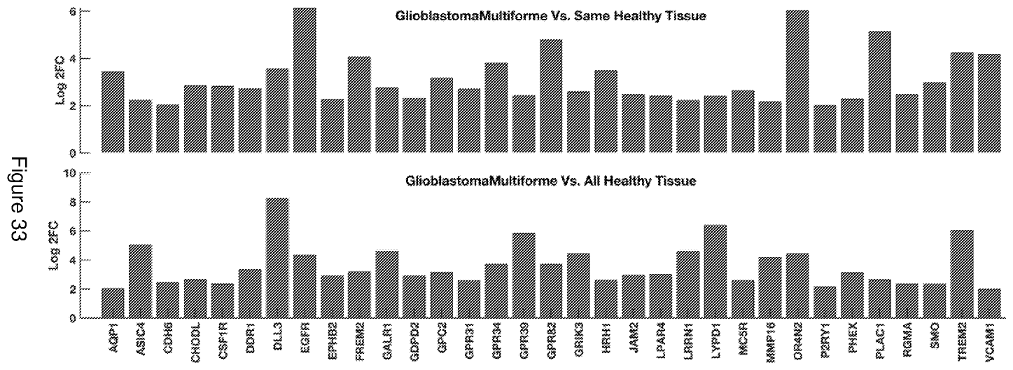

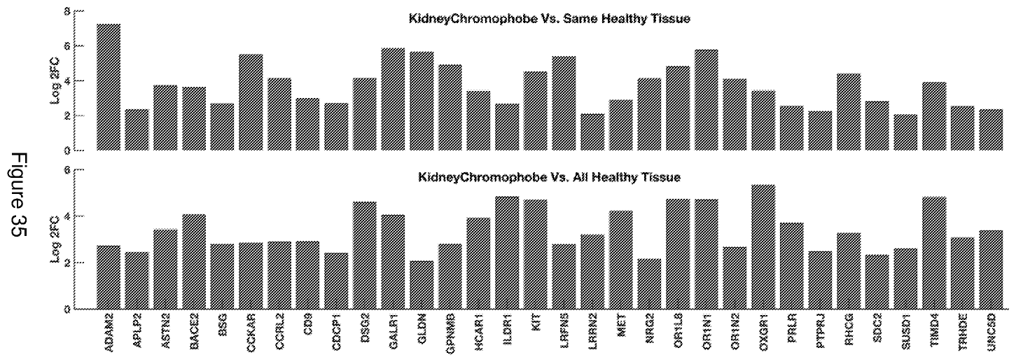

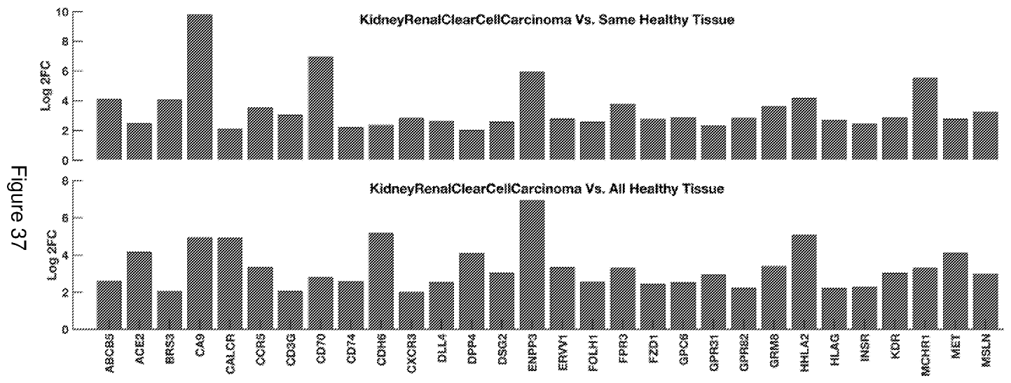

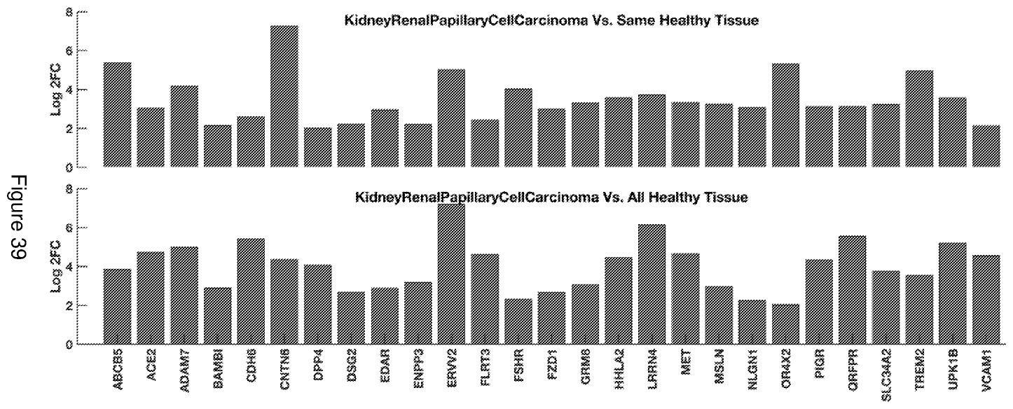

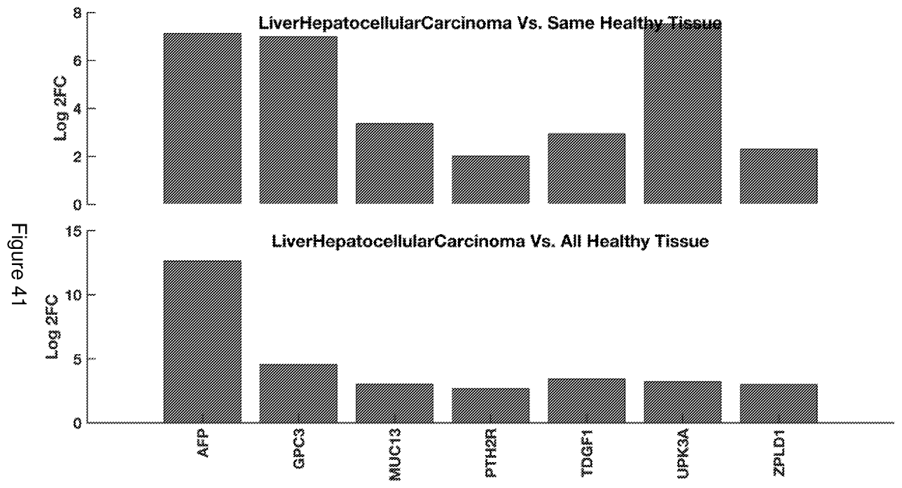

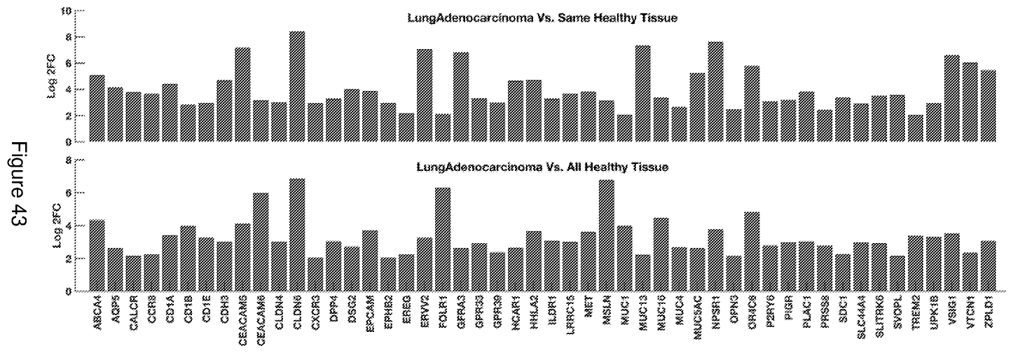

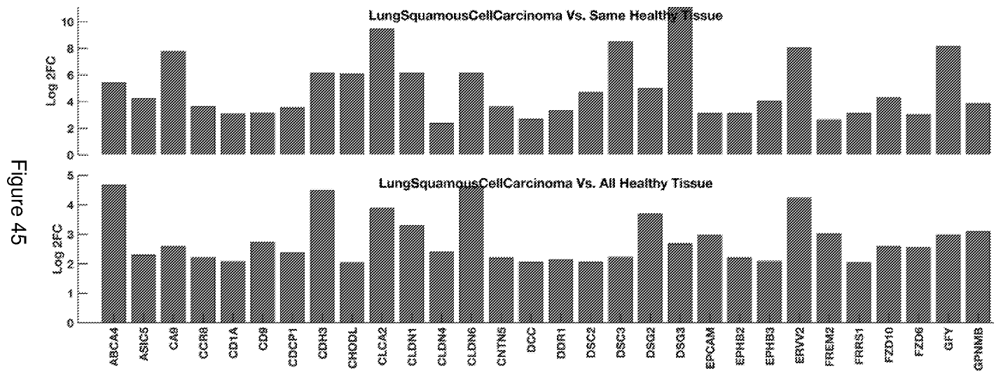

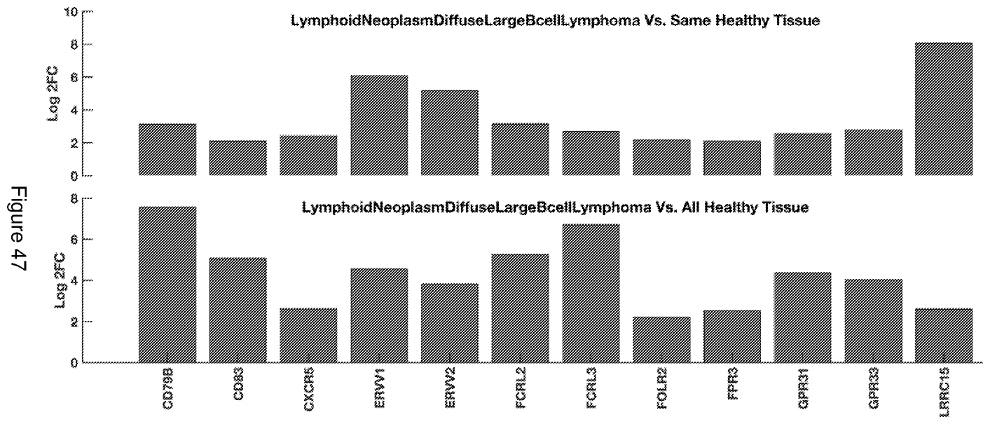

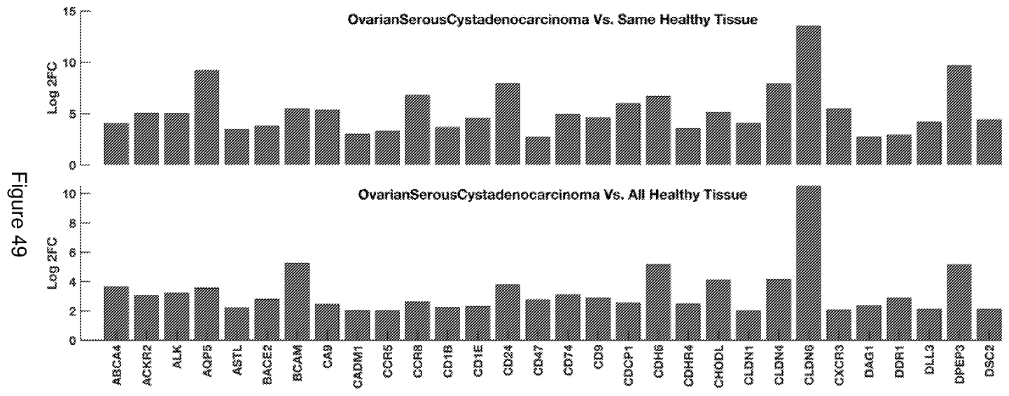

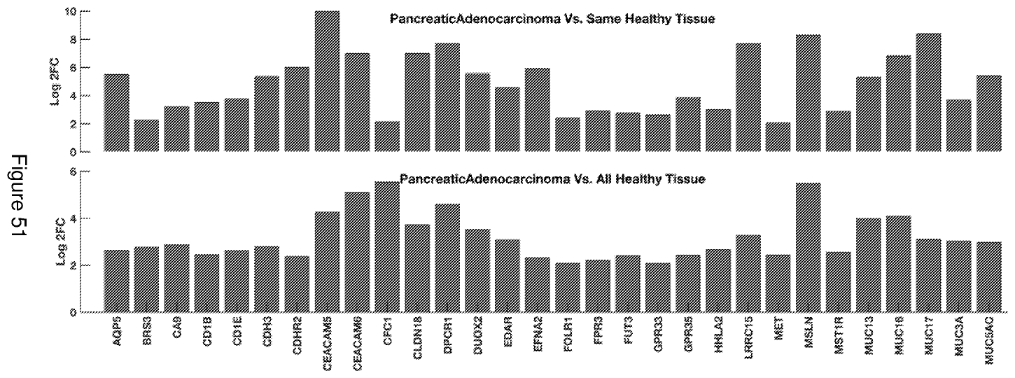

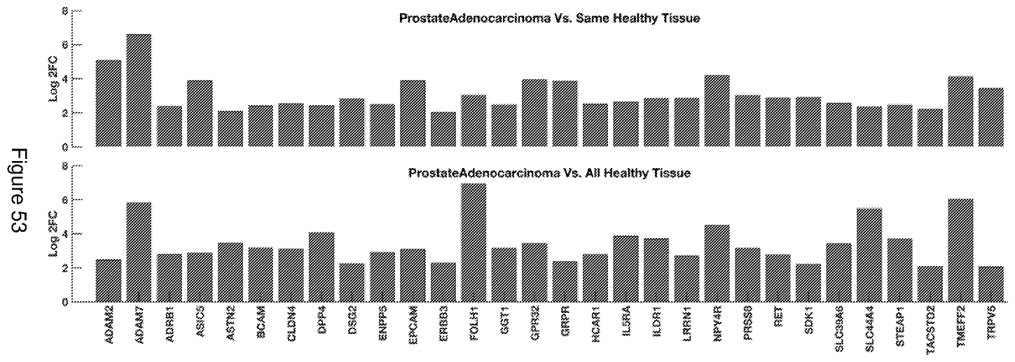

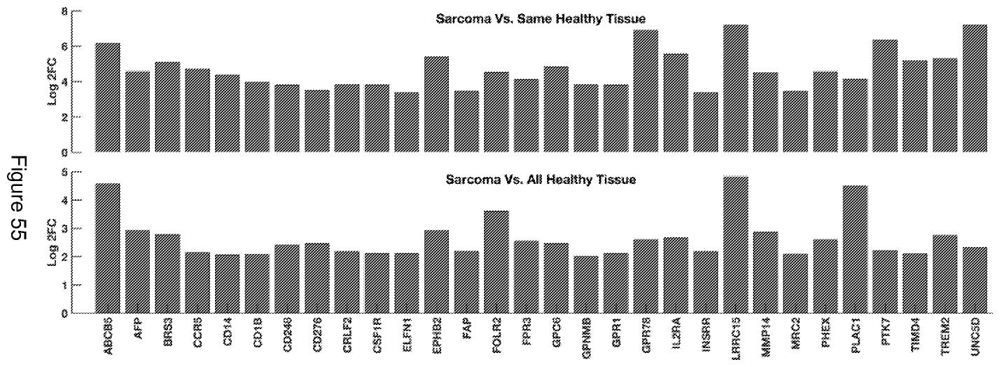

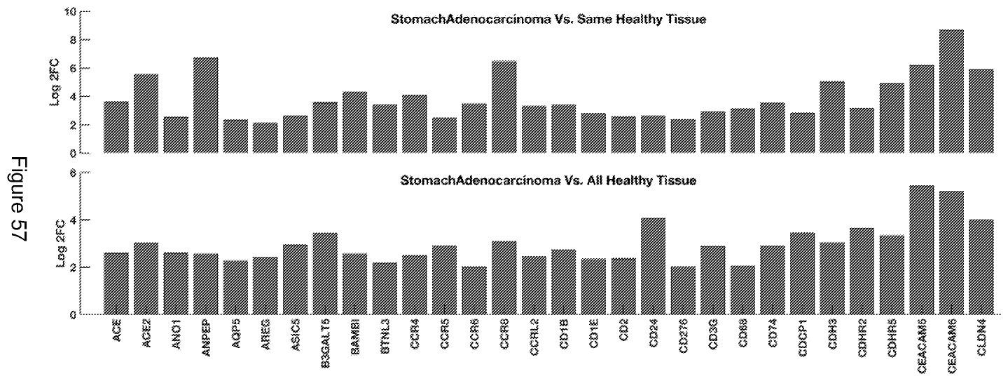

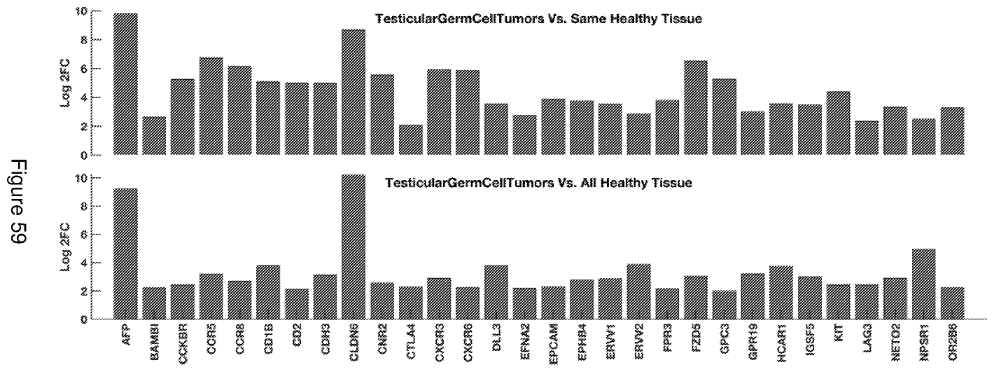

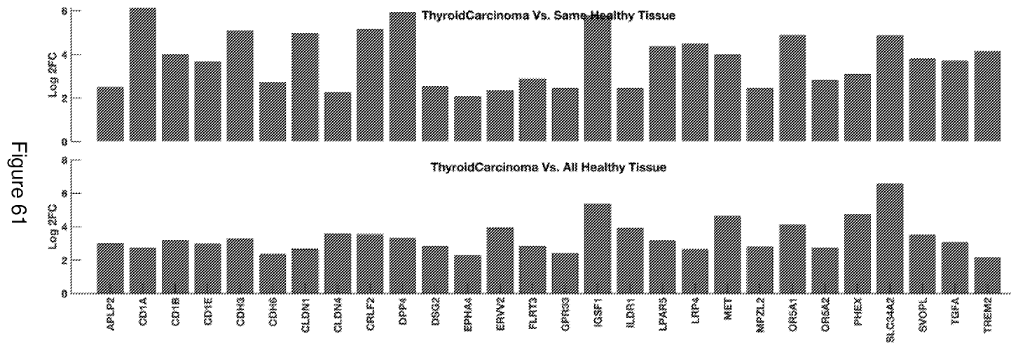

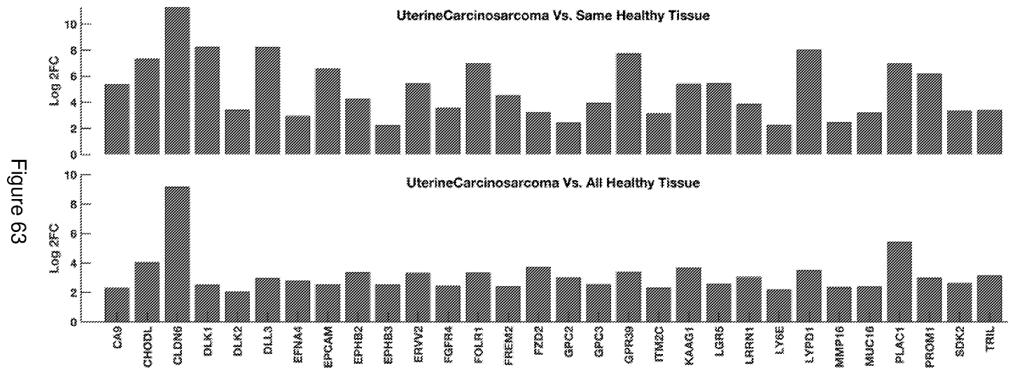

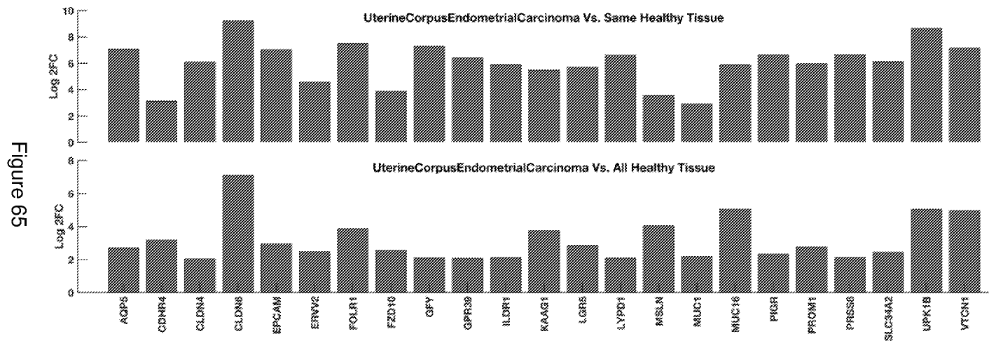

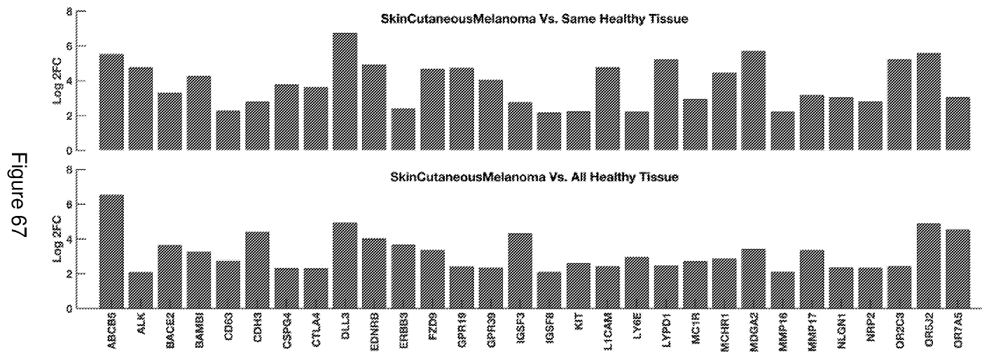

We return the CSR transcripts that we found upregulated (adjusted p-value<0.05 and log 2 fold change>2) on tumour cells in comparison to the healthy normal cells that the tumour cells originate from, but not being upregulated versus healthy tissue comparisons to yield what we propose as the “other” drug and antibody targets. These CSRs targets are shown in FIGS. 18, 20, 22, 24, 26, 28, 30, 32, 34, 36, 38, 40, 42, 44, 46, 48, 50, 52, 54, 56, 58, 60, 62, 64, 66 and 68. Anticancer agents targeted to these other targets can be administered locally in order to avoid any expected off target effects resulting from expression of these CSRs on other healthy tissues.

### REFERENCES

Anders S and Huber W. Differential expression analysis for sequence count data. Genome Biol 2010; 11:R106. doi:10.1186/gb-2010-11-10-r106
Ardlie K G, DeLuca D S, Segrè A V., Sullivan T J, Young T R, Gelfand E T, et al. The Genotype-Tissue Expression (GTEx) pilot analysis: Multitissue gene regulation in humans. Science (80-) 2015; 348:648-60. doi:10.1126/science.1262110.
Bausch-Fluck D, Hofmann A, Bock T, Frei A P, Cerciello F, Jacobs A, et al. A mass spectrometric-derived cell surface protein atlas. PLoS One 2015; 10. doi:10.1371/journal.pone.0121314.
Collado-Torres L, Nellore A, Kammers K, Ellis S E, Taub M A, Hansen K D, et al. Reproducible RNA-seq analysis using recount2. Nat Biotechnol 2017; 35:319-21. doi:10.1038/nbt.3838.
Cotto K C, Wagner A H, Feng Y-Y, Kiwala S, Coffman A C, Spies G, et al. DGldb 3.0: a redesign and expansion of the drug-gene interaction database. Nucleic Acids Res 2018; 46:D1068-73. doi:10.1093/nar/gkx1143.
Dai X, Cheng H, Bai Z, Li J. Breast Cancer Cell Line Classification and Its Relevance with Breast Tumour Subtyping. J Cancer 2017; 8:3131-41. doi:10.7150/jca.18457.
Fisher, B., J. Bryant, N. Wolmark, E. Mamounas, A. Brown, E. R. Fisher, D. L. Wickerham, M. Begovic, A. DeCillis, and A. Robidoux, Effect of preoperative chemotherapy on the outcome of women with operable breast cancer. Journal of Clinical Oncology, 1998. 16(8): p. 2672-2685.
Gene Ontology Consortium: going forward. NucleicAcids Res 2015; 43:D1049-56. doi:10.1093/nar/gku1179.
Gerber, D. E., Targeted therapies: a new generation of cancer treatments. American family physician, 2008. 77(3).
Kawaji H, Kasukawa T, Forrest A, Carninci P, Hayashizaki Y. The FANTOM5 collection, a data series underpinning mammalian transcriptome atlases in diverse cell types. Sci Data 2017; 4:170113. doi:10.1038/sdata.2017.113.
Lachmann A, Torre D, Keenan A B, Jagodnik K M, Lee H J, Wang L, et al. Massive mining of publicly available RNA-seq data from human and mouse. Nat Commun 2018; 9:1366. doi:10.1038/s41467-018-03751-6.
Liu, S. V., L. Melstrom, K. Yao, C. A. Russell, and S. F. Sener, Neoadjuvant therapy for breast cancer. Journal of surgical oncology, 2010. 101(4): p. 283-291.
Nguyen D-T, Mathias S, Bologa C, Brunak S, Fernandez N, Gaulton A, et al. Pharos: Collating protein information to shed light on the druggable genome. Nucleic Acids Res 2017; 45:D995-1002. doi:10.1093/nar/gkw1072.
Park, C. C., M. Mitsumori, A. Nixon, A. Recht, J. Connolly, R. Gelman, B. Silver, S. Hetelekidis, A. Abner, and J. R. Harris, Outcome at 8 years after breast-conserving surgery and radiation therapy for invasive breast cancer: influence of margin status and systemic therapy on local recurrence. Journal of Clinical Oncology, 2000. 18(8): p. 1668-1675.
Pawar A. CLASSIFICATION OF BREAST CANCER CELL LINES INTO SUBTYPES BASED ON GENETIC PROFILES 2015.
Platt J C, Platt J C. Probabilistic Outputs for Support Vector Machines and Comparisons to Regularized Likelihood Methods. Adv LARGE MARGIN Classif 1999:61-74.
Pontén F, Jirström K, Uhlen M. The Human Protein Atlas—a tool for pathology. J Pathol 2008; 216:387-93. doi:10.1002/path.2440.
Quiñonero-Candela J, Ramussen C E, Williams C K I. Approximation Methods for Gaussian Process Regression. 2007.
Recht, A., B. Silver, S. Schnitt, J. Connolly, S. Hellman, and J. R. Harris, Breast relapse following primary radiation therapy for early breast cancer. I. Classification, frequency and salvage. International Journal of Radiation Oncology* Biology* Physics, 1985. 11(7): p. 1271-1276.
UniProt: the universal protein knowledgebase. Nucleic Acids Res 2017; 45:D158-69. doi:10.1093/nar/gkw1099.
Weinstein J N, Collisson E A, Mills G B, Mills Shaw K R, Ozenberger B A, Ellrott K, et al. The Cancer Genome Atlas Pan-Cancer analysis project. Nat Publ Gr 2013; 45. doi:10.1038/ng.2764.
Yang W, Soares J, Greninger P, Edelman E J, Lightfoot H, Forbes S, et al. Genomics of Drug Sensitivity in Cancer (GDSC): a resource for therapeutic biomarker discovery in cancer cells. Nucleic Acids Res 2012; 41:D955-61. doi:10.1093/nar/gks 1111.
Zarin D A, Tse T, Williams R J, Carr S. Trial Reporting in ClinicalTrials.gov—The Final Rule. N Engl J Med 2016; 375:1998-2004. doi:10.1056/NEJMsr1611785.

1. A method of identifying an anticancer agent, the method comprising:
(i) establishing which cell surface receptors (CSR's) occur on the surface of a cell from a cancerous organ or tissue obtained from a subject;
(ii) establishing which CSR's occur on the surface of a cell from a healthy part of the same organ or tissue in (i);
(iii) determining which of the CSR's are differentially expressed in the cancerous organ or tissue compared to healthy organ or tissue;
(iv) identifying the differentially expressed CSR's as targets for anticancer therapy;
(vi) confirming that the differentially expressed CSR's are expressed at a higher level on the cancer cells as compared to the cells from the healthy part of the same organ or tissue in the subject or as compared to their expression in other healthy organs or tissues in the subject; and
(vii) identifying one or more anticancer agents which target one or more of the differentially expressed CSR's from the cancerous organ or tissue, which anticancer agents do not target cells from a healthy part of the same organ or tissue,
wherein the anticancer agent preferentially binds to the differentially expressed CSR on the surface of a cell from a cancerous organ or tissue, and
wherein the anticancer agent is preferentially administered systemically to the subject when it does not bind to a CSR on the surface of a cell from a healthy organ or tissue, and wherein the anticancer agent is preferentially administered locally to a tissue or organ of the subject when it does bind to a CSR on the surface of a healthy organ or tissue.

2. The method of claim 1, further comprising providing an organ or tissue sample collected from a subject for use in step (i).

3. The method of claim 1, wherein the presence of a CSR is established through measuring the mRNA transcript levels encoding the CSR in the cell.

4. The method of claim 3, wherein the mRNA transcript level is measured by microarray, SAGE, blotting, RT-PCR, sequencing or quantitative PCR.

5. The method of claim 1, wherein the anticancer agent is selected from the group consisting of a polynucleotide, protein, peptide or small molecule.

6. The method of claim 1, wherein the differentially expressed CSR in the cancerous organ or tissue is overexpressed by a log-fold difference, and wherein when the differentially expressed CSR is expressed at a log-fold difference of between 1 and 2 as compared to a cell from a healthy organ or tissue the anticancer agent is an anticancer agent which would be suitable for local administration to the cancerous organ or tissue, and wherein when the differentially expressed CSR is expressed on a cancerous cell at a log-fold difference of greater than 2 as compared to its expression on a cell from a healthy organ or tissue the anticancer agent is an anticancer agent which would be suitable for systemic administration to a subject.

7. The method of claim 6, wherein the differentially expressed CSR is either (i) not expressed in the healthy organ or tissue of the subject, or (ii) is expressed in a healthy organ or tissue of the subject at a level below a log 2 fold difference of 2.

8. The method of claim 1, wherein the subject is a human.

9. The method of claim 1, wherein the presence of the CSR on the surface of a cell from a cancerous organ or tissue and the absence of the CSR on the surface of cells from all healthy organs or tissues makes it a suitable target for the anticancer agent.

10. The method of claim 1, wherein the anticancer agent is selected based on its specificity to the differentially expressed CSR.
